(19) United States
(12) Patent Application Publication
(10) Pub. No.: US 2003/0153894 A1
(43) Pub. Date: Aug. 14, 2003

(54) Absorbent articles having improved stretchability

(76) Inventors: Bernadette Gibbs, Statham, GA (US); Betsy Johnson, Atlanta, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 09/988,064

(22) Filed: Nov. 16, 2001

(51) Int. Cl. A61F 13/15

(52) U.S. Cl. 604/386

(57) ABSTRACT

This disclosure describes an absorbent article, preferably a diaper, having improved stretchability. Specifically, a portion of the waist region of the absorbent article that contains a connection mechanism is stretched by applying a predetermined force to the waist region and connection mechanism. The waist region stretches more than about 125% of its original width when an 800 gram force is applied, and the connection mechanism stretches by more than about 200% of its original width, when an 800 gram force is applied. The absorbent article has improved flexibility and is capable of fitting a variety of wearer sizes.

### BACKGROUND OF THE INVENTION

1. Field of the Invention

The present invention relates to absorbent articles having improved stretchability. Specifically, the invention relates to absorbent articles whose backsheet can stretch more than about 125% of its original width, and whose connection mechanisms can stretch more than about 200% of its original length when an 800 gram force is applied to an about 4-inch wide laterally cut section of the waist region of the absorbent article. The absorbent articles of the invention have improved fitting characteristics and are capable of being used for a number of different sized wearers.

2. Description of Related Art

Disposable absorbent articles, such as diapers, sanitary products, incontinent pads, and the like have obtained wide acceptance by consumers. Such absorbent articles generally include a liquid permeable topsheet, a liquid impermeable backsheet for preventing body exudates from leaking out, and an absorbent member interposed there between for absorbing such body exudates. Conventional liquid impermeable backsheets usually provide satisfactory liquid impermeability, but these sheets typically are not flexible enough to closely conform to the shape of wearer's body, and consequently provide poor leakage protection. They also limit the ability of the absorbent article to stretch.

Infants and other incontinent individuals wear absorbent articles such as diapers to receive and contain urine and other body exudates. Absorbent articles function both to contain discharged materials and to isolate the materials from the body of the wearer and from the wearer's garments and bed clothing. Disposable absorbent articles having many different basic designs are known to the art. For example, Reissue U.S. Pat. No. 26,152, describes a conventional disposable diaper which has achieved worldwide acceptance and commercial success. Further, U.S. Pat. No. 5,246,433 discloses a unitary disposable absorbent article that can be used as a training pant. The disclosures of these documents are incorporated by reference herein in their entirety.

In the market today, the consumer has a number of different basic diaper designs to choose from depending on the desired options, comfort and cost. These diaper designs include conventional diapers, belted diapers, and “pull-on” type diapers or training pants. However, many of the absorbent articles on the market today are capable of fitting only a small range of wearer sizes and therefore, the consumer must continually monitor the size of the wearer to determine which diaper will comfortably and effectively fit the wearer. Thus, the consumer must purchase different diapers depending on the desired characteristics for the intended use. These various sizes typically are referred to as stages, and most diapers are sold in sizes ranging from Stage 1 (smallest) to Stage 6 (largest).

Conventional diaper designs generally are the least expensive type of absorbent article to produce and are generally acceptable for use on babies and persons who are sick or otherwise confined to a bed. A conventional diaper typically is fitted to the wearer by first placing a portion of the diaper under the wearer (generally, the back portion of the diaper is placed under the buttocks and rear waist of the wearer) and then pulling the remainder of the diaper through the wearer's legs. The rear portion of the diaper then is attached to the front portion of the diaper on each side of the wearer by a connection mechanism (tab containing tape or a hook/loop fastener).

Introducing a degree of stretchability into personal care products such as diapers, sanitary napkins, adult incontinent products and the like is believed to provide these products with certain benefits. For example, U.S. Pat. No. 2,866,459 describes a stretchable, reusable cotton diaper. Stretchability is imparted to the cotton fabric by the presence of longitudinal and transverse elastic stitching. U.S. Pat. No. 3,371,668 discloses a sanitary napkin employing a nonwoven fabric with machine direction elasticity as a cover. The described fabric comprises a nonwoven web of fibers having a plurality of elastic means, in extended condition, secured to the web.

U.S. Pat. No. 4,606,964 describes a bulked web composite and method of making the same, where the composite is formed by adhering a reticulated web of elastic material to at least one gatherable web. Upon release of the tensioning forces, the gatherable web is gathered by the reticulated web to form a bulked web composite. U.S. Pat. No. 4,847,134 discloses a stretchable absorbent undergarment, including an inner layer or topsheet, a liquid impervious outer layer or backsheet, an absorbent layer and a stretchable layer. The stretchable layer comprises a continuous layer of stretchable material that may be perforated. These perforations are said to render the elastomeric layer permeable.

U.S. Pat. No. 5,957,908 discloses an elastomeric ear panel used with “convertible” absorbent articles, whereby the ear panels (or flaps) are said to enable the use of the article as a conventional diaper, or as a “pull-on” diaper. The specifically described ear panels impart the stretchability characteristics described therein. U.S. Pat. No. 5,938,652 discloses an absorbent article having a selectively elasticized waist flap that forms a pouch to contain the body exudates. The specific waist flap described therein is said to conform better to the wearer's back during use, while the remainder of the absorbent article is positioned away from the wearer's back, thus forming a pouch.

U.S. Pat. No. 5,451,219 discloses a stretchable absorbent article whereby the liquid permeable topsheet is connected to the liquid impermeable backsheet in discrete and non-discrete regions thereby forming a series of longitudinal pleats having channels disposed therein. The absorbent article is described as capable of absorbing a desirable amount of liquids, and at the same time capable of being elastically stretchable and contractible to better conform to the wearer's body during use. U.S. Pat. No. 5,411,498 discloses a stretchable shaped absorbent garment containing at least two generally parallel elastomeric strands stretch bonded to the garment along its length. When the article is in a relaxed state, the elastomeric strands are reduced in length rendering the article stretchable.

The disclosures of all of the aforementioned United States patents are incorporated by reference herein in their entirety. Specifically, various elements, absorbent article configurations, and the like, are useful in the context of the present invention, as will be readily understood by one having ordinary skill in the art upon reading the remaining disclosure herein.

### SUMMARY OF THE INVENTION

It is a feature of an embodiment of the invention to provide absorbent articles that are more comfortable to the user, and that can adapt and stretch to fit more users. It is a feature of another embodiment of the invention to provide a method of measuring the stretchability of an absorbent article, and a method of measuring the stretchability of a connection mechanism disposed on the absorbent article.

In accordance with these and other features of the invention, there is provided an absorbent article including a topsheet, a backsheet, and an absorbent material disposed between the topsheet and the backsheet. The topsheet and backsheet each has a longitudinal front end and back end, which preferably are in substantial alignment with one another. The absorbent article also includes at least a back waist edge adjacent the back end of the topsheet and backsheet, a front waist edge adjacent the front end of the topsheet and backsheet, and side edges disposed between the front waist edge and the back waist edge. The article also includes a back waist region defined by the back waist edge and side edges, a front waist region defined by the front waist edge and side edges, and a crotch region disposed between the back waist region and the front waist region. At least the front waist region or the back waist region includes a connection mechanism, whereby the waist region that includes the connection mechanism stretches by more than 125% of its original width, when an 800 gram force is applied to an about 4-inch wide laterally cut portion of the waist region.

In accordance with an additional feature of an embodiment of the invention, there is provided an absorbent article including a topsheet, a backsheet, and an absorbent material disposed between the topsheet and the backsheet. The topsheet and backsheet each has a longitudinal front end and back end, which preferably are in substantial alignment with one another. The absorbent article also includes at least a back waist edge adjacent the back end of the topsheet and backsheet, a front waist edge adjacent the front end of the topsheet and backsheet, and side edges disposed between the front waist edge and the back waist edge. The article further includes a back waist region defined by the back waist edge and side edges, a front waist region defined by the front waist edge and side edges, and a crotch region disposed between the back waist region and the front waist region. At least the front waist region or the back waist region includes a connection mechanism (preferably a tab connector), whereby the connection mechanism stretches by more than about 200% of its original length, when an 800 gram force is applied to the connection mechanism attached to a portion of the waist region that has been laterally cut to comprise about a 4-inch wide cut portion of the waist region.

In accordance with an additional feature of the present invention, there is provided a method of measuring the stretchability of: (i) the waist region (preferably a backsheet) of an absorbent article; and (ii) a connection mechanism attached to a waist region of an absorbent article. The method includes the following: laterally cutting about a 4-inch longitudinal section of the waist region that includes a connection mechanism; flattening the longitudinal section of the waist region on a surface without substantially stretching the longitudinal section; measuring the dimensions of the longitudinal section of the waist region including the dimensions of the connection mechanism; attaching a first end of the connection mechanism or longitudinal section to a surface, and attaching the opposite end of the longitudinal section or a second end of the connection mechanism disposed on the opposite side of the longitudinal section from the first end to a second surface (preferably a weight); applying a force, preferably a force from about 700 to about 1500 grams, to the longitudinal section of the waist region including the connection mechanism to thereby stretch the longitudinal section and connection mechanism; measuring the dimensions of the stretched longitudinal section and connection mechanism; and calculating the stretchability of the longitudinal section, and the stretchability of the connection mechanism.

In accordance with yet another feature of an embodiment of the invention, there is provided an absorbent article as described above, whereby the absorbent article has a maximum stretchability, when about a 4-inch wide cut portion of the waist region of a number of different sizes are subjected to an 800 gram force, of greater than about 185% for newborn babies, of greater than about 175% for crawling babies, and of greater than about 175% for walking babies, whereby the maximum stretchability is determined in accordance with the following:

$$Max Stretch = \frac{{\sum }_{n = 1}^{n} ( Max Cirst / MnCirun ) \times 100}{n}$$

wherein:

MxCirstr is the stretched maximum circumference;

MnCirun is the unstretched minimum circumference; and

n is the number of stages of diapers in which the maximum stretchability is measured.

Newborn babies absorbent articles encompass articles in stages 0, 1, and 2 articles, crawling babies absorbent articles encompass articles in stages 3 and 4, and walking babies absorbent articles encompass articles in stages 5 and 6.

In accordance with yet another feature of an embodiment of the invention, there is provided an absorbent article as described above, whereby the absorbent article has a Stretchability Index (or SI) of greater than about 540 for newborn babies, of greater than about 515 for crawling babies, and of greater than about 505 for walking babies, where SI is the sum of the Maximum Stretchability values for each of the 800, 1,000, and 1,200 gram forces:

SI=MaxStretch₍₈₀₀₎+MaxStretch_{(1,000)}+MaxStretch_{(1,200)}

wherein:

MaxStretch₍₈₀₀₎ is MaxStretch for an 800 gram force;

MaxStretch_{(1,000)} is MaxStretch for a 1,000 gram force; and

MaxStretch_{(1,200)} is MaxStretch for a 1,200 gram force.

The various MaxStretch values are those obtained for newborn babies, crawling babies, and walking babies, and the sum of these values results in the Stretchability Index for the corresponding newborn babies, crawling babies, and walking babies. These and other features of the invention will be readily apparent to those skilled in the art upon reading the Detailed Description of Preferred Embodiments, in connection with the attached drawings that follow.

### BRIEF DESCRIPTION OF THE DRAWINGS

FIG. 1 is a plan view of an absorbent article.

FIG. 2 illustrates a plan view of a procedure whereby about a 4-inch wide longitudinal section of a waist region of an absorbent article is laterally cut.

FIG. 3 illustrates measuring the dimensions of a section of an absorbent article containing non-tape connection mechanisms.

FIG. 4 illustrates measuring the dimensions of a section of an absorbent article containing tape connection mechanisms.

FIG. 5 illustrates measuring the dimensions of a hook/loop-type connection mechanism.

FIG. 6 illustrates measuring the dimensions of a tape-type connection mechanism.

FIG. 7 depicts a connection mechanism having elastic materials disposed therein.

FIG. 8 depicts a variation of the connection mechanism of FIG. 7.

FIG. 9 illustrates attaching a portion of the waist region to an Instron device to measure the stretchability.

FIG. 10 illustrates an apparatus useful in measuring the stretchability of a backsheet and connection mechanism of an absorbent article.

FIG. 11 illustrates measuring the dimensions of the longitudinal section of an absorbent article while being stretched.

FIG. 12 is a plan view of an embodiment of an absorbent article having enhanced stretchability.

### DETAILED DESCRIPTION OF PREFERRED EMBODIMENTS

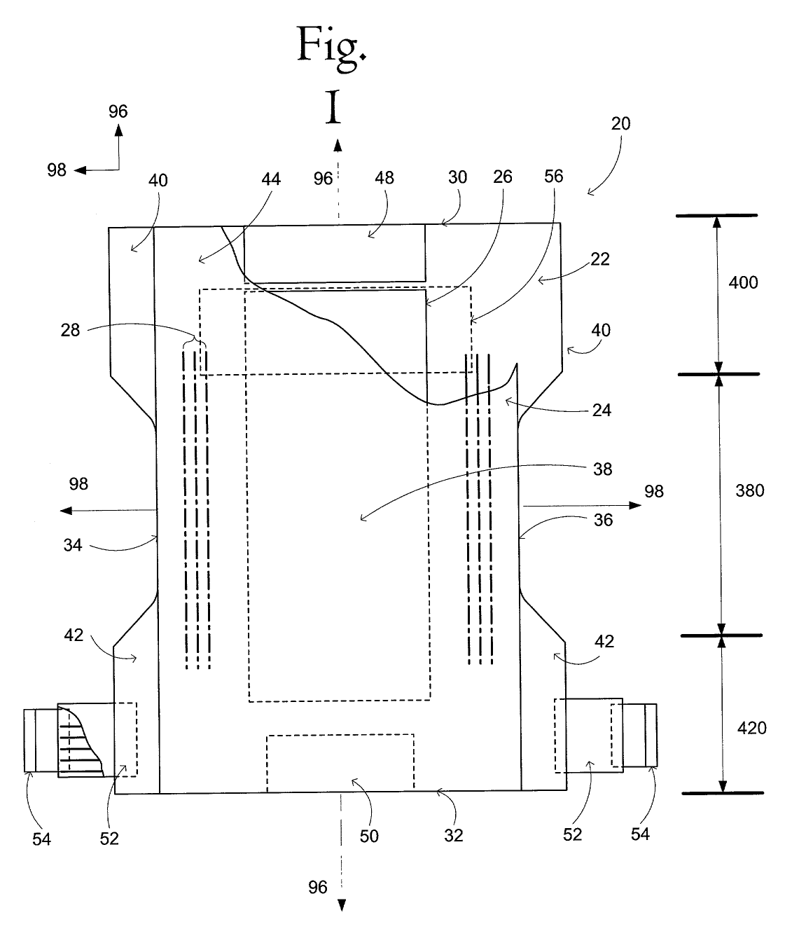

Throughout this description, the expressions “absorbent article,” and “absorbent garment” refer to articles that absorb and contain body exudates, and, more specifically, refer to articles that are placed against or in proximity to the body of the wearer to absorb and contain the various exudates discharged from the body. The term “disposable” as it is used herein, denotes absorbent articles that are not intended to be laundered or otherwise restored or reused as an absorbent article (i.e., they are intended to be discarded after a single use and, preferably, to be recycled, composted or otherwise disposed of in an environmentally compatible manner). A “unitary” absorbent article or absorbent garment denotes articles that are formed of separate parts united together to form a coordinated entity so that they do not require separate manipulative parts like a separate holder and liner. A particularly preferred embodiment of an absorbent article of the present invention is the disposable absorbent article, diaper 20, shown in FIG. 1.

Exemplary absorbent articles and absorbent garments include, but are not limited to, diapers, diaper covers, disposable diapers, training pants, feminine hygiene products, and adult incontinence products. The invention can be used with all of the foregoing classes of absorbent articles and garments, without limitation, whether disposable or otherwise. Furthermore, the invention will be understood to encompass, without limitation, all classes and types of absorbent articles and garments, including those described above.

As used herein, the term “diaper” refers to an absorbent article generally worn by infants and incontinent persons that is worn about the lower torso of the wearer. “Diaper” also denotes an absorbent article that is fastened about the waist of the wearer, and does not include training pants and other “pull-on” or “convertible” type garments. “Pull-on” garments are those garments that already are fastened about the waist and hence, can be donned simply by pulling them up the legs of the wearer. “Convertible” type garments are those garments that can be used as either a “pull-on” garment, or a conventional diaper that is fastened about the waist after placing on a user.

FIG. 1 illustrates an embodiment of an absorbent article 20 of a preferred embodiment of the present invention in its flat-out, uncontracted state (i.e., with elastic induced contraction pulled out). The preferred embodiment shown in FIG. 1 depicts an article 20 having a longitudinal dimension 96 and a lateral dimension 98. The article 20 also has two centerlines, a longitudinal centerline 96-96 and a lateral centerline 98-98. The term “longitudinal”, as used herein, refers to a line, axis, or direction in the plane of the article 20 that is generally aligned with (e.g. approximately parallel with) a vertical plane that bisects a standing wearer into left and right halves when the article 20 is worn. The term “lateral”, as used herein, refers to a line, axis or direction that lies within the plane of the article 20 that generally is perpendicular to the longitudinal direction (which divides the wearer into front and back body halves). In the preferred embodiment shown in FIG. 1, the upper half of the article 20 bisected by lateral centerline 98-98 is the front portion of the article, and the lower half of the article 20 bisected by lateral centerline 98-98 is the rear portion of the article.

A portion of the article 20 has been cut-away to more clearly show the construction of the article 20. The portion of the article 20 that faces the wearer, the garment facing surface 24, or topsheet 24, faces the viewer in FIG. 1. As shown in FIG. 1, the article 20 preferably comprises a liquid pervious topsheet 24, a liquid impervious backsheet 22 that preferably is joined to the topsheet, and an absorbent core 26 positioned between the topsheet 24 and the backsheet 22.

The encasement of the absorbent article elements listed above may be accomplished by a variety of mechanisms, each of which is well known to those skilled in the art. For example, the topsheet 24 and backsheet 22 may be joined to one another by bonding the respective layers using construction adhesives, or using ultrasonic bonding to join the layers. As used herein, the term “joined” encompasses configurations whereby an element is directly secured to the other element by affixing the element directly to the other element, and configurations whereby the element is indirectly secured to the other element by affixing the element to an intermediate member(s) which in turn are affixed to the other element. For clarity, FIG. 1 illustrates the article 20 with all of its elastic components, described in more detail herein, in their fully extended position. When the elastic components are in their relaxed or contracted positions, the garment will have shirred or wrinkled areas that can expand when force is applied in the plane of the article.

The garment 20 usually has a first waist edge 30, which may lie along the front waist of a wearer, and an opposed second waist edge 32, which may lie along the waist at the wearer's back. First waist edge 30 can be formed by the longitudinal front end (not shown) of the topsheet 24, and the longitudinal front end (not shown) of the backsheet 22. Second waist edge 32 can be formed by the longitudinal back end (not shown) of the topsheet 24, and the longitudinal back end (not shown) of the backsheet 22. Two opposed side edges 34, 36 define leg encircling openings when the garment is fitted on a wearer, and the first waist edge 30 is attached to the second waist edge 32. Thus, with the first waist edge 30 to the front of a wearer, side edge 34 is the left leg side edge and side edge 36 is the right leg side edge. Lying between the opposed side edges 34, 36 is a crotch region 38, which is central to the garment.

As illustrated in the embodiment of FIG. 1, the outer layer 22 may extend outwardly from each of the opposed side edges 34, 36 along the first and second laterally extending waist edges 30, 32. In this manner, a pair of first waist extensions or “ears” 40 can be formed extending from each side edge along the first waist edge 30. A pair of second waist extensions or “ears” 42 may be formed extending from each side edge along the second waist edge 32. The inner layer 24 may extend partially or completely over the waist extensions 40, 42 or it may terminate just beyond the outer boundary of the absorbent core 26 along an outer boundary 44. The outer boundary 44 is the region in which the inner sheet 24 is bonded or joined to the outer sheet 22 to encase the absorbent core 26.

Thus, the layers of the garment 20 as illustrated in FIG. 1, may have an hourglass shape. The top and bottom of the hourglass shape form first and second waist regions of the garment, with the first waist portion comprising each of the first waist extensions 40 and the area of the diaper there between. This first (or front) waist region is designated by numeral 400 in FIG. 1. The second waist region comprises the second waist extensions 42 and the region of the garment along the second waist edge 32 between extensions 42. This second (or rear) waist region is designated by numeral 420 in FIG. 1. The opposed sides of the hourglass shape form the opposed side edges 34, 36. The crotch region 38 of the garment 20 is central to the hourglass shape, and has a narrower width than the waist portions. Crotch region 38 also is designated by numeral 380 in FIG. 1. Those skilled in the art will appreciate that garment 20 may have a rectangular shape, with no waist extensions 40, 42. As yet another alternative, the garment 20 may have a “T” shape, having only a pair of first waist extensions 40, or a pair of second waist extensions 42.

The leg openings defined by the opposed side edges 34 and 36 preferably are gathered and elasticized to create a better fitting garment that is more capable of retaining bodily wastes. There are a variety of methods that are well known in the art for providing elasticized leg openings by incorporating elastic materials into the garment 20 between the outer layer 22 and the inner layer 24 along each of the opposed side edges 34, 36. The elastic material can be in the form of longitudinal strands 28, webbing, strips, or any other functional configuration. Any suitable elastic material may be used, such as rubber, elastic foams, Spandex (which is commercially available from E. I. DuPont Nemours & Company, a business with its corporate headquarters in Wilmington, Del.), or LYCRA® (also available from E. I. DuPont Nemours & Company).

In one embodiment of the invention, the elastic material is expanded and then attached to the garment using elastic adhesives, such that when the elastic relaxes or contracts the material of the garment forms expandable shirrs or gathers in the side edges 34 and 36. In another embodiment of the invention a heat-shrinkable elastomeric material, of the type that is relatively unstable and inelastic when unshrunk, and relatively stable and elastic when shrunk, is incorporated into the garment and subsequently heat shrunk to form expandable shirrs or gathers in the side edges 34 and 36. Those skilled in the art are capable of designing and utilizing a suitable elastic material or materials to form a comfortably sealable leg opening defined by side edges 34 and 36.

In one embodiment, the invention includes a first waist elastic 48 in the boundary area 44 adjacent to the first (or front) waist edge 30, and a second waist elastic 50 in the boundary area adjacent to the second (or rear) waist edge 32. The waist elastics 48 and 50 can be formed by placing an elastic material, such as an expandable foam or a heat-shrinkable elastomeric material, between the outer layer or backsheet 22 and the inner layer or topsheet 24. Such a construction may be completed as described above regarding the elasticized leg openings. Although the garment 20 illustrated in FIG. 1 has both a first waist elastic 48 and a second waist elastic 50, it is anticipated that an embodiment of the invention can have a waist elastic, as described above, in only one of the waist regions 400, 420 or in neither of the waist regions 400, 420. It will be understood from the following description, however, that various types of elastic materials, as well as various types of stretchable topsheet 24, backsheet 22 and absorbent core 26 materials may be used in the invention to render the absorbent garment 20 stretchable in accordance with the present invention.

The garment 20 preferably has two connection mechanisms 52 preferably protruding laterally from the second waist extensions 42 near the second (or rear) waist edge 32. In the preferred embodiment shown in FIG. 1, the connection mechanism is shown as two separate connection mechanisms 52, which in turn are preferably tabs. The connection mechanism may be comprised of a single belt-type material, however, instead of two separate tabs. As shown in FIG. 1, each tab 52 preferably has a gripping connector 54 located at or near its laterally outboard end. The garment is fitted to the wearer by attaching the gripping connectors 54 to a target connector 56 located on the backsheet 22 near the first waist edge 30. Accordingly, it is particularly preferred in the invention that garment 20 be a diaper, as that term is defined above to exclude pull-on type garments and convertible garments. Although the embodiment of the invention depicted in FIG. 1 has a single target connector 56, it is apparent that the invention could also be practiced using a plurality of target connectors attached to the backsheet 22, the topsheet 24, or both, and that the connection mechanism may consist of a one-piece construction extending laterally across waist region 420. Furthermore, it should be clear that embodiments of the invention may be practiced with the connector tabs 52 preferably located near the first waist edge 30, and the target connector located near the second waist edge 32.

In a preferred embodiment of the present invention, the absorbent article 20 comprises a topsheet 24 and a backsheet 22 which have length and width dimensions generally larger than those of the absorbent core 26. The topsheet 24 and the backsheet 22 preferably extend beyond the edges of the absorbent core 26 to thereby form the periphery of the article 20. While the topsheet 24, the backsheet 22, and the absorbent core 26 may be assembled in a variety of well known configurations, exemplary configurations are described generally in U.S. Pat. No. 3,860,003, and U.S. Pat. No. 5,151,092, each of which is incorporated herein by reference in its entirety.

The absorbent core 26 may be any absorbent member that is generally compressible, conformable, non-irritating to the wearer's skin, and capable of absorbing and retaining liquids such as urine and other certain body exudates. As shown in FIG. 1, the absorbent core 26 is rectangular in shape, although an embodiment of the article 20 may have an asymmetric, modified T-shaped absorbent core 26 having the top of the “T” in the front waist region 400 but a generally rectangular shape in the rear waist region 420. It is apparent that the absorbent core 26 may be manufactured in a wide variety of sizes and shapes (e.g., rectangular, hourglass, asymmetric, etc.) and from a wide variety of liquid-absorbent materials commonly used in disposable diapers and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials. The fluff component may also be mixed with distributed particles of superabsorbent material, with the resulting core 26 being preferably surrounded by a tissue over-wrap, or other material, to contain the superabsorbent particles. The moisture absorbent layer 26 may extend for the entire length and width of the garment 20, but alternatively it may extend only partially along the length and width of the garment.

The configuration and construction of the absorbent core 26 may vary (e.g., the absorbent core may have varying caliper zones, a hydrophilic gradient, a superabsorbent gradient, or lower average density and lower average basis weight acquisition zones; or may comprise one or more layers or structures). Further, the size and absorbent capacity of the absorbent core 26 may also be varied to accommodate wearers ranging from infants through adults. However, the total absorbent capacity of the absorbent core 26 should be compatible with the design loading and the intended use of the article 20. Exemplary absorbent structures for use as the absorbent core 26 of the present invention that have achieved wide acceptance and commercial success are described in U.S. Pat. Nos. 4,610,678, 4,673,402, 4,888,231, and 4,834,735, the disclosures of which are incorporated by reference herein in their entirety. The absorbent core may further comprise a dual core system containing an acquisition-distribution core of chemically stiffened fibers positioned over an absorbent storage core, as described in U.S. Pat. No. 5,234,423, and in U.S. Pat. No. 5,147,345, the disclosures of which are incorporated herein by reference in their entirety.

The backsheet 22 preferably is positioned adjacent the garment-facing surface of the absorbent core 26 and preferably is joined thereto by attachment mechanisms such as those well known in the art. For example, the backsheet 22 may be secured to the absorbent core 26 by a uniform continuous layer of adhesive, a patterned layer of adhesive, or an array of separate lines, spirals, or spots of adhesive. Suitable adhesives include, for example, HL-1258 by H. B. Fuller Company of St. Paul, Minn.; Findley 2031 and H2587-01 by Ato Findley Inc. of Wauwatosa, Wis.; and NS34-5665 by National Starch Co. of Bridgewater, N.J.

An example of a suitable attachment mechanism is one that comprises an open pattern network of filaments of adhesive, as disclosed in U.S. Pat. No. 4,573,986. Another suitable attachment mechanism is one that comprises several lines of adhesive filaments swirled into a spiral pattern, as illustrated by the apparatus and methods shown in U.S. Pat. Nos. 3,911,173, 4,785,996, and 4,842,666. Each of these patents is incorporated herein by reference in its entirety. Alternatively, the attachment mechanism may comprise heat bonds, pressure bonds, ultrasonic bonds, dynamic mechanical bonds, or any other suitable attachment mechanism or combinations of these attachment mechanisms as are known in the art. Embodiments of the present invention also are contemplated wherein the absorbent core is not joined to the backsheet 22, the topsheet 24, or both in order to provide greater extensibility throughout the article 20.

As mentioned above, backsheet 22 preferably is impervious to liquids (e.g., urine) and preferably is manufactured from a thin plastic film, although other flexible and stretchable liquid impervious materials may be used. As used herein, the term “flexible” refers to materials that are compliant and will readily conform to the general shape and contours of the human body. Flexible also denotes a material having a degree of stretchability. The backsheet 22 prevents the exudates absorbed and contained in the absorbent core 26 from wetting articles that contact the article 20 such as bedsheets and undergarments. However, the backsheet 22 preferably is breathable so as to permit vapors to escape from the absorbent core 26 while still preventing exudates from passing through the backsheet 22. Thus, the backsheet 22 preferably comprises a woven or nonwoven material, polymeric films such as thermoplastic films of polyethylene or polypropylene, or composite materials such as a film-coated nonwoven material. A suitable backsheet 22 is a thermoplastic film having a thickness of from about 0.012 mm (0.5 mil) to about 0.051 mm (2.0 mils) joined with a nonwoven covering comprising natural or synthetic fibers. If the backsheet 22 is a film, it preferably is embossed and/or matte finished to provide a more clothlike appearance.

At least a portion of the backsheet 22 may be subjected to mechanical stretching in order to provide both a “zero strain” stretch laminate that forms the portion of the backsheet 22 coinciding with the waist region 420. The backsheet 22 can be prestrained by directing the backsheet through an incremental mechanical stretching system similar to the operation described with respect to the formation of the “zero strain” stretch laminate backsheet and elasticized ear panels in U.S. Pat. No. 5,151,092, the disclosure of which is incorporated by reference herein in its entirety. It is preferred in the invention that backsheet 22 be stretchable to the extent that it permits at least a portion of the waist region 420 (about a 4-inch thick laterally cut portion) stretch more than 125% of its original width when a force of about 800 grams is applied.

Any mechanism capable of imparting the desired stretchability can be used in the present invention. For example, a series of elastic elements may be disposed within the matrix of backsheet material 22, or disposed between backsheet 22 and topsheet 24 in the lateral (98) and longitudinal (96) direction. In addition, the backsheet 22, or any portion thereof, may comprise a structural elastic-like film (SELF) web. A structural elastic-like film web is an extensible material that exhibits an elastic-like behavior in the direction of elongation without the use of added elastic materials. Such materials are disclosed in, for example, U.S. Pat. No. 5,957,908, the disclosure of which is incorporated by reference herein in its entirety.

The strainable web material is preferably comprised substantially of linear low density polyethylene (LLDPE). The strainable web material may also be comprised of other polyolefins such as polyethylenes, including low density polyethylene (LDPE), ultra low density polyethylene (ULDPE), high density polyethylene (HDPE), or polypropylene and blends thereof with the above and other materials. Examples of other suitable polymeric materials that may also be used include, but are not limited to, polyester, polyurethanes, compostable or biodegradable polymers, heat shrink polymers, thermoplastic elastomers, and breathable polymeric structures.

Other components that may be added to backsheet 22, or incorporated therein, include fluted elastic, scrim, additional elastics, and stretchable adhesives. It is preferred to include these materials in both the backsheet 22 and the topsheet 24 so that the entire absorbent article 20 is stretchable to the same degree. Stretchable adhesives could be woven into the backsheet 22 or topsheet 24 materials using techniques know in the art.

It is preferred in the present invention that backsheet 22, together with the other components of article 20 be capable of being stretched to the same degree as the about 4-inch portion of waist region 420 or 400. It also is preferred that the materials be capable of being stretched in both the longitudinal (96) and lateral (98) dimensions so that the entire article 20 may be fully and completely stretched to accommodate different sized wearers. The respective components may be manufactured to have the desired stretchability. For example, various polymer films can be manufactured to include larger portions of flexible, rubber-like components, such as propylene impact copolymer films containing a higher portion of ethylene/propylene rubber. Alternatively, the respective components can be modified to impart the desired flexibility, such as by incorporation of various types of elastic materials, scrim elastics, or foam materials that are capable of being stretched. Using the guidelines and stretching characteristics provided herein, a person skilled in the art is capable of fabricating a suitable backsheet 22 and/or topsheet 24.

The topsheet 24 preferably is positioned adjacent the body-facing surface of the absorbent core 26 and is preferably joined thereto and to the backsheet 22 by any attachment mechanism well known in the art. Suitable attachment mechanisms are described above with respect to joining the backsheet 22 to the absorbent core 26. In a preferred embodiment of the present invention, the topsheet 24 and the backsheet 22 are joined directly to each other in the article periphery areas denoted by numeral 44 in FIG. 1. The topsheet 24 is preferably compliant, soft feeling, and non-irritating to the wearer's skin. For example, topsheet 24 may be treated with a variety of solutions or lotions to impart skin wellness and diaper rash-preventing characteristics to the material, such as those disclosed in U.S. Pat. No. 5,938,649, the disclosure of which is incorporated by reference herein in its entirety.

Further, the topsheet 24 preferably is liquid pervious permitting liquids (e.g., urine) to readily penetrate through its thickness. A suitable topsheet 24 may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet 24 preferably is comprised of a hydrophobic material to isolate the wearer's skin from liquids that have passed through the topsheet 24 and are contained in the absorbent core 26 (i.e. to prevent rewet). If the topsheet 24 is made of a hydrophobic material, at least the upper surface of the topsheet 24 should be treated to render it hydrophilic so that liquids will transfer through the topsheet 24 more rapidly. This diminishes the likelihood that body exudates will flow off the top sheet 24 rather than being drawn through the topsheet 24 and being absorbed by the absorbent core 26.

The topsheet 24 preferably is rendered hydrophilic by treating it with a surfactant. Suitable methods for treating the topsheet 24 with a surfactant include spraying the topsheet 24 material with the surfactant and immersing the material into the surfactant. A more detailed discussion of such a treatment and hydrophilicity is contained in U.S. Pat. No. 4,988,344 and U.S. Pat. No. 4,988,345, each of which is incorporated by reference herein in its entirety. During treatment with the surfactant, other components may be applied to topsheet 24 to render it softer, or to render diaper rash-preventing characteristics thereto. For example, various ingredients can be applied together with the surfactant, such as vitamin E, aloe, lubricants, and other skin wellness ingredients.

There are a number of manufacturing techniques that may be used to manufacture the topsheet 24. For example, the topsheet 24 may be a nonwoven web of fibers. When the topsheet 24 comprises a nonwoven web, the web may be spunbonded, carded, wet-laid, melt-blown, hydroentangled, combinations of the above, or the like. A suitable topsheet 24 is carded and thermally bonded by means well known to those skilled in the art. A satisfactory topsheet 24 comprises staple length polypropylene fibers having a denier of about 2.2. As used herein, the term “staple length fibers” refers to those fibers having a length of at least about 15.9 mm (0.625 inches). Preferably, the topsheet 24 has a basis weight from about 14 to about 25 grams per square meter. A suitable topsheet is manufactured by Veratec, Inc., a Division of International Paper Company, of Walpole, Mass. under the designation P-8.

When commercial materials are used to make topsheet 24, it is preferred that they be treated to render them more stretchable. It is preferred in an embodiment of the present invention that at least a portion of the topsheet 24 be manufactured or treated in a manner similar to backsheet 22 described above, so that both sheet materials have similar degrees of stretchability. For example, elastic materials may be disposed within the topsheet 24 matrix, or sandwiched between topsheet 24 and backsheet 22. Other techniques known to impart enhanced stretching characteristics may be used so long as they provide the preferred and desirable features of the present invention.

The article 20 preferably further comprises elasticized leg cuffs (not shown) that typically are disposed adjacent side edges 34 and 36. Elastic elements 28 typically are disposed in or near side edges 34, 36 to produce the leg gathers, as described above. The leg cuffs are prepared in a similar manner by disposing various elastic elements in between sheets of material so that the cuffs are shirred when in a relaxed state. Leg cuffs generally provide improved containment of liquids and other body exudates. Each elasticized leg cuff may comprise several different embodiments for reducing the leakage of body exudates in the leg regions. (The leg cuff can be and is sometimes also referred to as leg bands, side flaps, barrier cuffs, stand-up cuffs, or elastic cuffs.). For example, U.S. Pat. No. 3,860,003 describes a disposable diaper that provides a contractible leg opening having a side flap and one or more elastic members to provide an elasticized leg cuff (gasketing cuff). U.S. Pat. No. 4,909,803 describes a disposable diaper having “stand-up” elasticized flaps (barrier cuffs) to improve the containment of the leg regions. U.S. Pat. No. 4,695,278 describes a disposable diaper having dual cuffs including a gasketing cuff and a barrier cuff. U.S. Pat. No. 4,704,115 discloses a disposable diaper or incontinence garment having side-edge-leakage-guard gutters configured to contain free liquids within the garment. Each of these patents is incorporated herein by reference in its entirety. While each elasticized leg cuff may be configured so as to be similar to any of the leg bands, side flaps, barrier cuffs, or elastic cuffs described above, each elasticized leg cuff preferably comprises a gasketing cuff as described in the above-referenced U.S. Pat. No. 3,860,003 and a barrier cuff as described in the above-referenced U.S. Pat. No. 4,909,803.

The article 20 preferably further comprises at least one waist elastic 50 (or 48) that helps provide improved fit and containment. The expression “waist elastic” as it is used herein, denotes an element having elastic properties, such that it can be stretched in the lateral direction (98), the longitudinal direction (96), or both. The waist elastic 50 may be elastically extensible or inelastic. In preferred embodiments, the waist elastic 50 has at least some ability to expand and contract in conjunction with wearer's motions. The waist elastic 50 preferably extends longitudinally outwardly from at least one of the edges of the absorbent core 26 and generally forms at least a portion of the waist edges 32, 30 of the article 20.

Disposable diapers are generally constructed so as to have two waist features, one positioned in the rear waist region 420 (e.g., waist elastic 50) and one disposed in the front waist region 400 (e.g., waist elastic 48), although diapers can be constructed with a single waist feature. Further, while the waist elastic 50 or any of its constituent elements can comprise a separate element affixed to the article 20, the waist elastic 50 may be constructed as an extension of other elements of the diaper such as the backsheet 22, or the topsheet 24, preferably both the backsheet 22 and the topsheet 24.

The waist elastic 50 preferably comprises an elastic material suitable for application in a garment to be fitted to a wearer, such as any suitable stretchable or elastomeric material. Suitable elastomeric materials for use as the waist elastic 50 are described below. The waist elastic 50 may be constructed in a number of different configurations including those described in U.S. Pat. No. 4,515,595, U.S. Pat. No. 5,151,092; each of these references being incorporated herein by reference in their entirety. Further, the waist elastic 50 may comprise “pleats” that may be activated by the wearer to provide additional extension in the waist regions.

Various embodiments of the invention contemplate stretchable absorbent articles 20 whereby the stretchability of the waist region 420 that includes the connection mechanism 52 is greater than 125%, when an 800 gram force is applied to an about 4-inch wide laterally cut portion of the waist region. Each of the components of article 20 described above cooperate with one another to impart the desired stretchability. It is preferred that topsheet 24, backsheet 22, absorbent core 26, and waist elements 48, 50 be designed to provide the requisite stretching characteristics. Various elastic materials may be disposed or otherwise intertwined within or between these components to impart the desirable characteristics.

It also is preferred that the stretchability of waist region 420 that includes the connection mechanism 52 be greater than 130% when a 1,000 gram force is applied to an about 4-inch wide laterally cut portion of the waist region. In addition, it is preferred that the stretchability of waist region 420 that includes the connection mechanism 52 be greater than 130% when a 1,200 gram force is applied to an about 4-inch wide laterally cut portion of the waist region. Stretchability is meant to include the amount the waist region stretches when the predetermined amount of force is applied, relative to its initial unstretched dimension.

One elastically extensible material that has been found to be especially suitable for use in article 20 is a laminate of at least one coverstock layer (e.g., non-woven or woven film) joined with an elastomeric film. Examples of suitable coverstock materials are hereinbefore discussed with respect to the topsheet 24 and the backsheet 22. Other suitable coverstock materials include nonwovens such as Fiberweb E004203 available from Fiberweb North America of Simpsonville, S.C., and Veratec 7pt., P-8 and P-14 available from Veratec Nonwoven Group of the International Paper Company of Walpole, Wash. Examples of suitable elastomeric films include Clopay 2870, a styrene block copolymer available from the Clopay Corporation of Cincinnati, Ohio, and Exxon 550 available from the ExxonMobil Corporation of Houston, Tex.

Foam materials may be used and disposed between or intertwined with the respective components described above. For example, suitable synthetic foams include: a) crosslinked natural rubber foams preferably having a caliper of approximately 50 mils and a density of approximately 13.3 pounds per cubic foot (0.214 grams per cubic cm), such as is available from Fulflex Inc., of Middletown, R.I., or as available from Ludlow Composites Corporation of Freemont, Ohio and marketed under the tradename Baby Foam; b) polyurethane foams having a caliper of approximately 80 mils and a density of approximately 2.06 pounds per cubic foot (0.033 grams per cubic cm), such as is available from Bridgestone of Yokohama, Japan and marketed under the tradename Bridgestone SG Polyurethane Foam; or c) polyurethane foam materials made from aliphatic isocyanates generally available from, for example, BASF Corporation, Germany. Other suitable materials include structural elastic-like film (SELF) webs, as described above, natural rubber, natural rubber foams, elastomeric scrims, woven or nonwoven elastomeric webs, elastomeric composites such as elastomeric nonwoven laminates, zero strain stretch laminates, prestrained stretch laminates or the like.

The topsheet 24, backsheet 22, absorbent core 26, and waist elastic 48, 50 also may comprise a laminate including an elastomeric scrim material. In one such preferred embodiment, the laminate of a scrim is laminated between nonwovens, films, scrims, laminates or any combinations thereof. Suitable nonwovens include those mentioned above, as well as the carded polypropylene RMS 66265 available from Veratec Nonwoven Group of the International Paper Company of Walpole, Wash.; and the spunbond polyethylene available from Corovin GmbH of Germany under the trade name COROLIND®. A suitable scrim is available from Conwed Plastics, Minneapolis, Minn., under the trade name REBOUND ELASTOMERIC NETTING®, having between 2 and 20 strands per inch extending in the machine direction and the cross-direction. In yet another embodiment, the respective components may be comprised of a laminate including a formed film such as X-15301 manufactured by Tredegar Film Products, Inc. of Terre Haute, Ind.

Other preferred materials for use in the invention are laminates made with elastomeric scrim (TN2510) from Conwed Plastics. The laminate typically consists of 3-5 layers, including the scrim. The layers other than the scrim preferably comprise nonwovens, films or apertured films. The laminate may be bonded by any means known in the art for joining layers of a laminate. Examples of suitable bonding means include, but are not limited to, heat, pressure, ultrasound, adhesive, cohesive and coextrusion. In one especially preferred embodiment, the laminate comprises differential bond regions that can be achieved by varying the amount or intensity of the bonding means throughout the laminate.

Another feature of the present invention is that the stretchability of the connection mechanism (connector tabs 52 shown in FIG. 1) is greater than about 200%, when an 800 gram force is applied to an about 4-inch wide laterally cut portion of the waist region 420. The same materials used to impart desirable stretching characteristics to topsheet 24, backsheet 22, absorbent core 26, and waist elements 48, 50 may be used to impart similar characteristics to connector tab 52. Preferably, the stretchability of the connection mechanism is greater than about 200%, when a 1,000 gram force is applied to an about 4-inch wide laterally cut portion of the waist region 420. In addition, the stretchability of the connection mechanism is greater than about 210%, when a 1,200 gram force is applied to an about 4-inch wide laterally cut portion of the waist region 420. Stretchability again is used in this context to denote the amount the connection mechanism stretches, when the predetermined force is applied, relative to its initial, unstretched dimension.

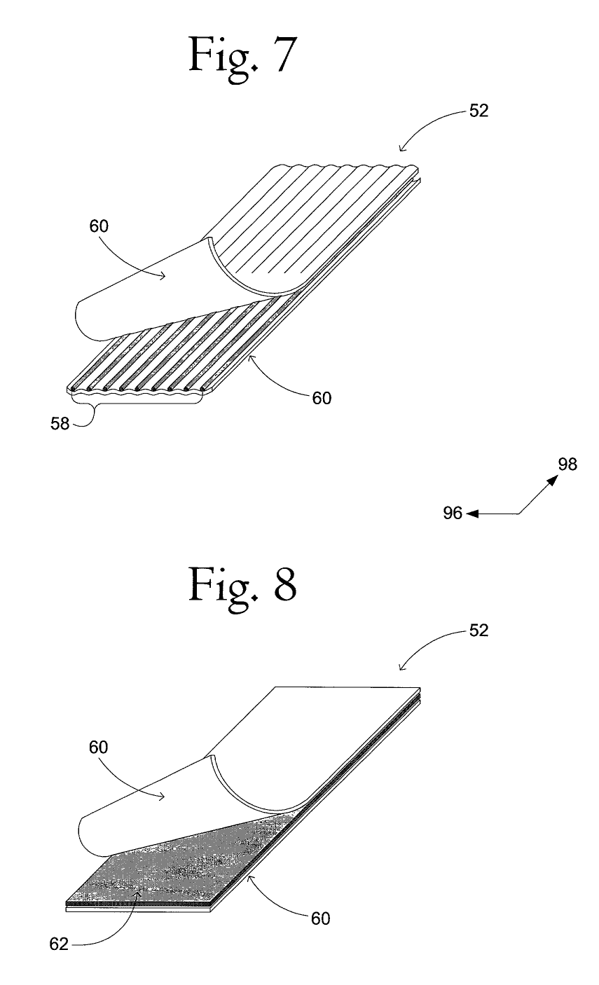

FIG. 7 illustrates an embodiment of a connector tab 52 in the fully-extended position. The tab 52 preferably is comprised of a plurality of elastic elements 58 disposed between two layer elements 60. For clarity, one layer element is depicted after being partially peeled away from the plane of the tab. The layer elements can be made from a number of materials, such as any of those described above. In one embodiment of the invention, the layer elements are made from spun-bonded nonwoven polypropylene material.

In the embodiment depicted in FIG. 7, the elastic elements 58 preferably are strands of elastic material, oriented roughly parallel with the lateral axis 98 of the article 20, which are attached to one or both of the layer elements 60 such that the layer elements 60 are shirred or gathered when the elastic elements 58 are in a relaxed or contracted position. The elastic elements 58 can be made from any number of elastic or elastomeric materials, such as rubber, spandex, or LYCRA, or any of the other materials described above with reference to topsheet 24, backsheet 22, absorbent core 26, and waist elements 48, 50. In a preferred embodiment, the elastic elements 58 are attached to the layer elements 60 using an elastic adhesive, and the layer elements 60 are attached to one another using the same elastic adhesive. The means of attachment preferably prevents the elastic elements 58 from becoming tangled or misaligned and maintains the wide flat shape of the tab 52, which ensures that the article 20 is operative, comfortable, and appears tidy.

Although the above preferred embodiment is exemplary of one method of attaching the elastic elements 58 to the layer elements 60 and attaching the layer elements 60 to one another, it is anticipated that the elastic elements 58 can be attached by any number of means. In addition, the means of attachment may be either continuous throughout the entire area of the connector tabs 52, or intermittent and dispersed.

FIG. 8 illustrates another embodiment of a tab 52 in the fully extended position. Again, for clarity, one layer element 60 is depicted after being partially peeled away from the plane of the tab. In this embodiment, a single elastic element 62 is disposed between two layer elements 60. The elastic element 62 can be comprised of a film of any suitable elastic material, including rubber, spandex, LYCRA and elastic polymers, a foam material, or any of the other materials described above with reference to topsheet 24, backsheet 22, absorbent core 26, and waist elements 48, 50. The elastic element 62 also may be comprised of a multidirectional elastic aggregate such as elastic webbing, netting, or scrim elastic, such as FLEXCEL™ Elastic Nonwoven Laminate, available from Kimberly-Clark Corporation, headquartered in Neenah, Wisc. The elastic element 62 can be attached to the layer elements 60 by any functional means, including elastic adhesives, as described above. Film, webbing, netting, and scrim elastic elements provide restoring forces in all planar directions, which may provide improved resistance to torsional forces in the plane of the article 20.

The connector tabs 52 also can be made from commercially available elastomeric materials such as Fabriflex®, available from Tredegar Film Products, headquartered in Richmond, Va. Such products are typically comprised of an elastic film layer sandwiched between nonwoven spun-bonded materials or similar constructions.

It is preferable to provide a single garment 20 that can be fitted to users with a variety of body sizes. Additionally, the garment's user or the user's caregiver should be able to unfasten and refasten the garment multiple times without reducing the utility of the garment. To meet these requirements, the tabs should be able to be extended and contracted many times without losing their elasticity.

It is conventional to provide disposable absorbent garments in various stages of development, with stages ranging from 0 to 6, with 0 being newly born babies, and 6 being the largest to accommodate large babies just prior to potty training. It is preferred in the invention to provide one absorbent garment 20 for newborn babies, one diaper for crawling babies, and one diaper for walking babies, thereby reducing the number of garments for all stages of babies by more than ½. Throughout this description, newborn babies correspond to stages 0, 1, and 2, crawling babies correspond to stages 3 and 4, and walking babies correspond to stages 5 and 6.

The elastic tabs 52 grip the user's torso and hold the garment in place. Ideally, the tabs will exhibit enough force to prevent the escape of bodily waste that is captured in the garment, but will also allow unrestricted movement and will not cause the user undue discomfort. The amount of gripping force exerted by the tabs preferably is a function of the spring constant of the elastic tabs. The spring constant of the tabs 52 may be varied along the longitudinal dimension 96 or lateral dimension 98 of the tab, making the article 20 more easily stretched in certain areas, which may be done to improve the fit, comfort, or sealing performance of the article 20.

Connector tabs 50 may include any connection mechanism capable of providing the above-described connecting function. Preferably, the connection mechanism is comprised of gripping connectors 54, and target connector 56, which may be a single target connector, or a plurality of target connectors. The fastening system may comprise any attachment mechanism known in the art including pressure sensitive adhesives, cohesive materials, mechanical fasteners such as hook and loop type fasteners, or any combination of these or any other attachment means known in the art. Exemplary adhesive tape tab fastening systems are disclosed in U.S. Pat. Nos. 3,848,594, and 4,662,875. Exemplary fastening systems comprising mechanical fastening components are described in U.S. Pat. Nos. 5,058,247, 4,869,724, and 4,846,815. An example of a fastening system having combination mechanical/adhesive fasteners is described in U.S. Pat. No. 4,946,527. Each of these patents is incorporated herein by reference in their entirety.

In a preferred configuration of the present invention, the fastening system comprises hook and loop type fasteners. As used herein, the term “hook and loop type fasteners” refers to fastening mechanisms that comprise a “hook” component (hereinafter referred to as gripping connector 54) and a complementary loop component (hereinafter referred to as target connector 56). The term “hook” is used to designate a material having engaging elements. Thus, the hook fastening material may also be referred to as a male fastener. It should also be understood that the use of the term “hook” should be non-limiting in the sense that the engaging elements may comprise any shapes as are known in the art so long as they are adapted to engage a complementary landing component.

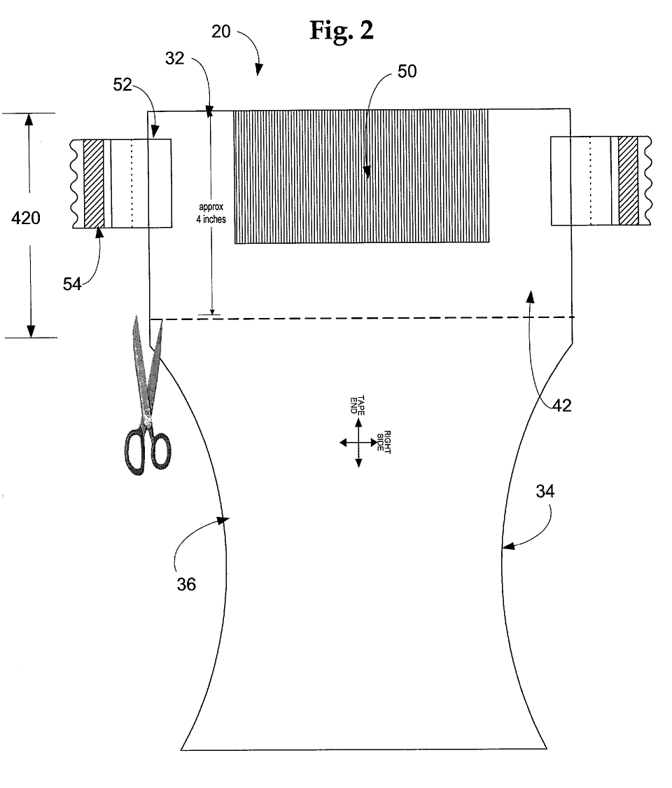

Another preferred embodiment of the present invention is a method of measuring the stretchability of the waist region 420 that includes the connection mechanism 52 by, for example, applying a predetermined force (e.g., from about 700 to about 1500 grams) to an about 4-inch wide laterally cut portion of the waist region 420. This method also is capable of measuring the stretchability of the connection mechanism (connector tabs 52 shown in FIG. 1) by, for example, applying a predetermined force (e.g., from about 700 to about 1500 grams) to an about 4-inch wide laterally cut portion of the waist region 420. The testing methods used to measure the stretchability will be described in more detail below, with specific reference to FIGS. 2-6, and FIGS. 9-11.

In accordance with the method of the invention, a sample from an absorbent article 20 must first be obtained and prepared. The method contemplates different measuring techniques used, depending on the particular type of fastening mechanism 52 employed, (e.g., whether the mechanism is a hook and loop or a tape fastener). Referring again to FIG. 1, and to FIG. 2, on the non-tape/tab end of the diaper, (area at or near waist region 400) measure and record the target tape/loop length and width (e.g., length and width of target connector 56). A portion of the waist region 420 then can be procured by cutting all the way through article 20 at approximately 4 inches from waist edge 32 so that all components in waist region 420 are intact, as shown in FIG. 2. In this regard, it is preferable to avoid cutting through waist elastic 50, or extensions 42. It also is preferable that portions of article 20 not be stretched while cutting.

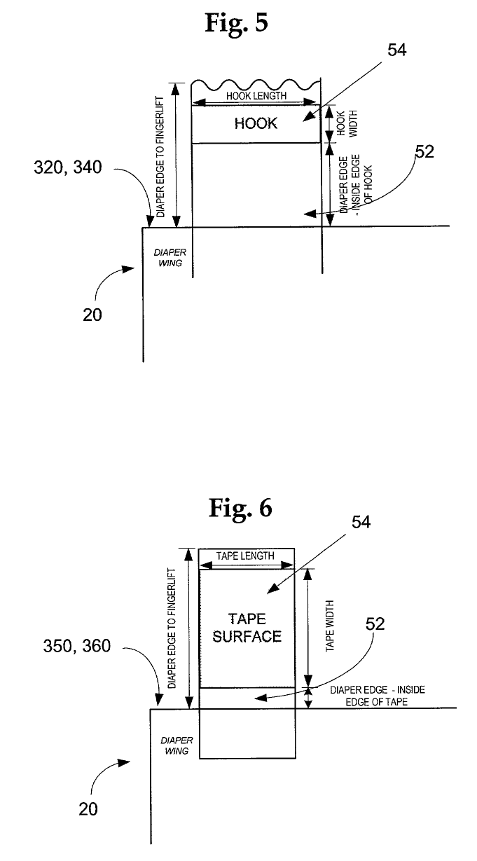

The dimensions of the cut-away portion can be measured, by first measuring the length and width of the connection tabs 52. For connection tabs 52 that include hook type gripping connector 54, FIG. 5 illustrates the various measurements to record. For connection tabs 52 that include a tape gripping connector 54, FIG. 6 illustrates the various measurements to record.

The cut-away portion of article 20, which corresponds in general to waist region 420 should be placed on a relatively hard, flat surface, with the backsheet 22 facing the surface, and topsheet 24 facing the viewer. side down). Without substantially stretching, open the connection mechanisms 52, and use relatively heavy weights 300 to hold the unstretched portion flat in place. Using a ruler, measure in a straight line across the portion of article 20 at or about the center of the connection mechanisms 52.

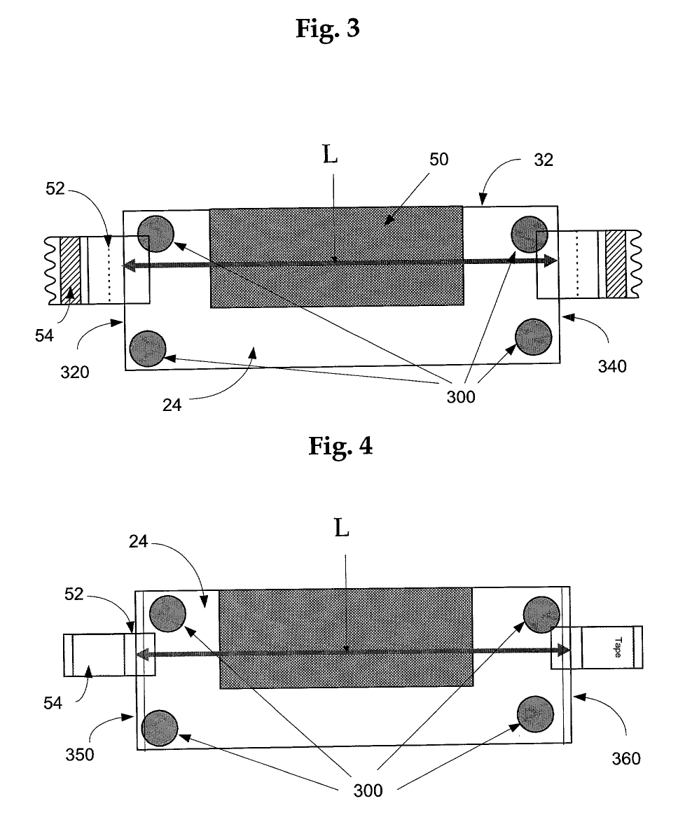

FIG. 3 illustrates the method of measuring the width of backsheet material for an article 20 that has non-tape connection mechanisms 52, such as hook gripping connectors 54. Turning now to FIG. 3, measure and record the distance from the left edge of the backsheet material 320 to the right edge of the backsheet material 340, making sure that the backsheet material is straight and not folded over. The measurement is denoted by the letter “L” in FIG. 3.

FIG. 4 illustrates the method of measuring the width of backsheet material for an article 20 that has tape connection mechanisms 52, such as gripping connectors 54 being comprised of an adhesive material. Turning now to FIG. 4, measure and record the distance from the tape crease on the left edge 350 to the tape crease on the right edge 360. The measurement is denoted by the letter “L” in FIG. 4.

Referring again to FIGS. 5 and 6, while the portion of article 20 is lying flat, measure and record for both left and right connection mechanisms 52, the distance from the edge of the article (320, 340 or 350, 360) to the inside edge of the gripping connector 54, albeit a hook connector or a tape connector. FIG. 5 shows the measurement for non-tape connection mechanisms 52, whereby one measures from the edge of the article 320, 340 to the innermost edge of the hook fastening material. FIG. 6 shows the measurement for tape connection mechanisms 52, whereby one measures from the tape crease edge 350, 360 to the inside edge of the exposed tape adhesive.

The distance from the edge of the article to the end of the connection mechanism 52 (e.g., the “fingerlift”) also is measured for both the right and left connection mechanisms. FIG. 5 illustrates the measurement for non-tape connection mechanisms 52, whereby one measures from the edge of the article 320, 340 to the outermost edge of the connection mechanism 52. FIG. 6 shows the measurement for tape connection mechanisms 52, whereby one measures from the tape crease edge 350, 360 to the outermost edge of the connection mechanism 52.

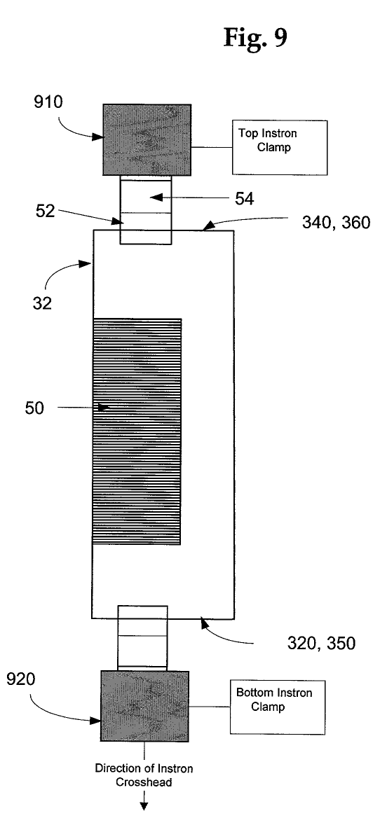

The measurements of the portion of article 20 while in a stretched state also need to be taken to determine the stretchability of the article. In one preferred embodiment of the present invention, one can use an Instron to measure the stretched dimensions. Preferred Instron measuring devices include Instron Modell 1122, and Modell 6500, available from Instron Corporation, Canton, Mass. Use of such a device is illustrated in FIG. 9. The Instron instrument should be calibrated to read up to about 1,500 g with a crosshead speed of about 12 in/min, making sure that the Return, Min and Max Limits do not interfere with testing.

Turning now to FIG. 9, the right connection mechanism 52 extending from the edge (340, 360) of article 20 is secured into the top clamp 910 of the instron. Make sure that none of the waist elastic elements 50, which are shown to the left in FIG. 9, are clamped into the instron. The portion of the article 20 should now simply hang from the top clamp 910. Next, the bottom clamp 920 of the instron should be lowered until it is even with the end of the left connection mechanism 52 extending from the edge (320, 350) of article 20. The left connection mechanism 52 then can be secured into the bottom clamp 920 of the instron, and the bottom clamp 920 lowered until the portion of article 20 is straight—until just before it is stretched. Those skilled in the art will appreciate that when the portion of article 20 is stretched, there is a tension reading on the instron. Accordingly, the test should be started with a tension reading of 0, thereby indicating no stretching.

The extension measurement then can be re-set to 0, and the crosshead lowered until the tension reading is at the predetermined force (e.g., 800, 1000, or 1,200 g), at which point the crosshead is stopped. This indicates the lateral stretching force. While in the stretched state, measure in a straight line across the diaper at the center of the tapes/tabs, as shown in FIGS. 3 and 4, and as described above with respect to the unstretched portion of article 20.

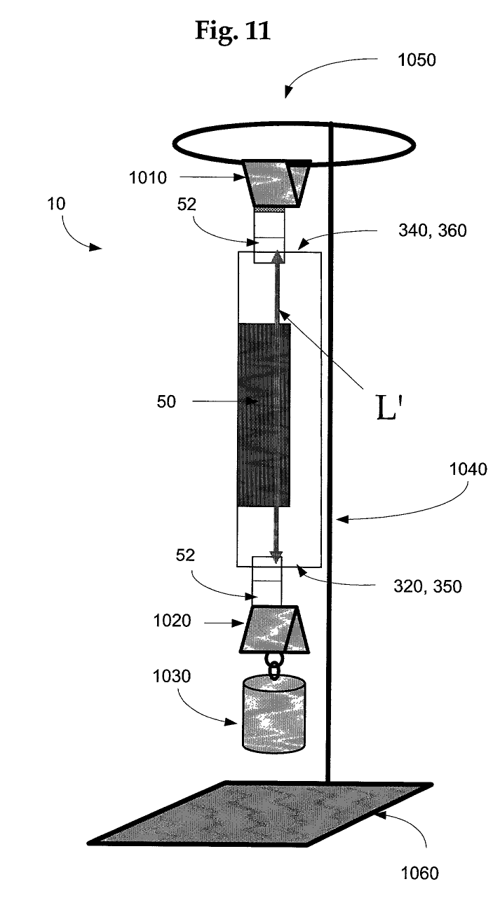

Specifically, for the non-tape connection mechanism 52, measure and record the distance from the left edge of the backsheet material 320 to the right edge of the backsheet material 340, making sure that the backsheet material is straight and not folded over. The measurement is denoted by the letter “L” in FIG. 3, but when in the stretched state, this width is designated by L′ (see, FIG. 11). For the tape connection mechanism 52, measure and record the distance from the tape crease on the left edge 350 to the tape crease on the right edge 360. The measurement is denoted by the letter “L” in FIG. 4, but when in the stretched state, this width is designated by L′ (see, FIG. 11).

The dimensions of connection mechanisms 52 also are measured in the stretched state. Again, FIGS. 5 and 6 illustrate how the measurements preferably are taken. FIG. 5 shows the measurement for non-tape connection mechanisms 52, whereby one measures from the edge of the article 320, 340 to the innermost edge of the hook fastening material. FIG. 6 shows the measurement for tape connection mechanisms 52, whereby one measures from the tape crease edge 350, 360 to the inside edge of the exposed tape adhesive.

The distance from the edge of the article to the end of the connection mechanism 52 (e.g., the “fingerlift”) also is measured for both the right and left connection mechanisms in the stretched state. Referring again to FIG. 5, this figure illustrates the measurement for non-tape connection mechanisms 52, whereby one measures from the edge of the article 320, 340 to the outermost edge of the connection mechanism 52. FIG. 6 shows the measurement for tape connection mechanisms 52, whereby one measures from the tape crease edge 350, 360 to the outermost edge of the connection mechanism 52.

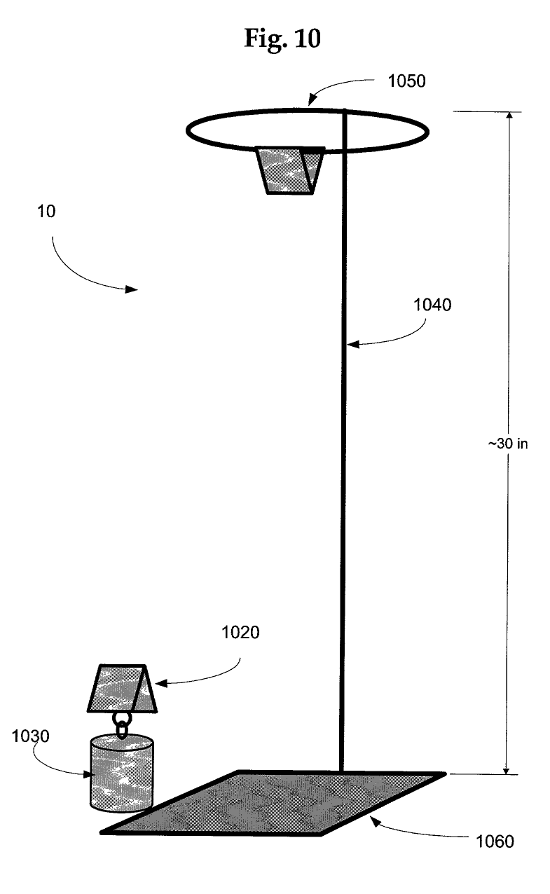

In another preferred embodiment of the invention, the dimensions of the about 4-inch wide laterally cut portion of the article 20 in the stretched state can be measured simply by suspending a predetermined (800, 1,000, or 1,200 gram) weight from the portion. An apparatus suitable for carrying out the stretching procedure is shown in FIG. 10. As shown therein, apparatus 10 includes a ring portion 1050, a base portion 1060, and a supporting bar 1040, which is about 30 inches or more in length. A top clamp 1010 is attached to ring portion 1050, and is used to attach one side of the portion of article 20 to apparatus 10. A bottom clamp 1020 is attached to the other side of the portion of article 20, and also has attached thereto a weight 1030 indicating the amount of force applied to the portion of article 20.

Referring now to FIG. 11, the right connection mechanism 52 may be hung from the top clamp 1010, making sure that none of the waist elastic elements 50 are clamped. The left connection mechanism 52 should then be hanging freely. From the left connection mechanism 52, hang the bottom claim 1020, and the predetermined weight 1030. Slowly and gently release the weight, so as not to let it swing or drop. The dimensions of the stretched portion of article 20 now can be measured. It is preferred to take measurements within 30 seconds so that additional stretching of the portion of article 20 corresponding generally to waist region 420 does not occur.

The dimensions of the stretched portion of article 20 are taken in the same manner as described above, with respect to the Instron apparatus. Specifically, for the non-tape connection mechanism 52, measure and record the distance from the left edge of the backsheet material 320 to the right edge of the backsheet material 340, making sure that the backsheet material is straight and not folded over. The measurement is denoted by the letter L′ in FIG. 11. For the tape connection mechanism 52, measure and record the distance from the tape crease on the left edge 350 to the tape crease on the right edge 360. The measurement is denoted by the letter L′ in FIG. 11.

The dimensions of connection mechanisms 52 also are measured in the stretched state. Again, FIGS. 5 and 6 illustrate how the measurements preferably are taken. FIG. 5 shows the measurement for non-tape connection mechanisms 52, whereby one measures from the edge of the article 320, 340 to the innermost edge of the hook fastening material. FIG. 6 shows the measurement for tape connection mechanisms 52, whereby one measures from the tape crease edge 350, 360 to the inside edge of the exposed tape adhesive.

The distance from the edge of the article to the end of the connection mechanism 52 (e.g., the “fingerlift”) also is measured for both the right and left connection mechanisms in the stretched state. Referring again to FIG. 5, this figure illustrates the measurement for non-tape connection mechanisms 52, whereby one measures from the edge of the article 320, 340 to the outermost edge of the connection mechanism 52. FIG. 6 shows the measurement for tape connection mechanisms 52, whereby one measures from the tape crease edge 350, 360 to the outermost edge of the connection mechanism 52.

After taking and recording the appropriate dimensions, the stretchability of the article 20 and connection mechanisms 52 can be calculated as follows. The overall width of the portion of article 20 can be calculated by adding the length of the two connection mechanisms 52 to the total length of the connection mechanism 52 in the stretched and unstretched states:

Tab-Tab Width: (measurement of the article 20 width from tab 52 end to tab 52 end)

Unstretched Width=width L+distance from (Right+Left) edges 320, 340, 350, or 360 to fingerlift (unstretched);

Stretched Width=width L′+distance from (Right+Left) edges 320, 340, 350, or 360 to fingerlift (stretched).

The maximum circumference of article 20 essentially is the width of the portion of article 20 (designated by L for unstretched or L′ for stretched), plus the distance from edges 320, 340, 350, or 360 on the right and left side to the inside edge of the gripping connector 54, plus the width of the target connector 56, which was measured before the article was cut transversely about 4-inches from waist edge 32. The maximum circumference therefore is the tab-to-tab stretched width (when stretched with the predetermined force), minus the width of gripping connectors 54 plus the lateral width of the target connector 56. This maximum circumference therefore assumes that gripping connectors 54 are attached to target connector 56 at the lateral-most edges of target connector 56 (the edges of target connector 56 that are closest to side edges 34 and 36, as shown in FIG. 1). The maximum circumference in the stretched condition does not take into consideration the stretchability of target connector 56, since the waist region 400 where target connector 56 is disposed is not stretched. Those skilled in the art will recognize, however, that in operation target connector 56 will stretch upon application of force from the attached connection mechanisms, thereby rendering the maximum circumference of garment 20 greater than the amount reported herein.

The maximum circumference can be calculated as follows:

Maximum Circumference: (the circumference of the waist of article 20 at the widest tab positioning on the target connector 56):

Unstretched Max. Circumference=width L+distance from (Right+Left) edges 320, 340, 350, or 360 to inside edge of hook/tape (unstretched)+target connector width 56.

Stretched Max. Circumference=width L′+distance from (Right+Left) edges 320, 340, 350, or 360 to inside edge of hook/tape (stretched)+target connector width 56.

The minimum circumference of article 20 essentially is the width of the portion of article 20 (designated by L for unstretched or L′ for stretched), plus the distance from edges 320, 340, 350, or 360 on the right and left side to the inside edge of the gripping connector 54, plus the width of the right and left hook and/or tape gripping connectors 54 (or 2× the width of gripping connector 54 width). This minimum circumference therefore assumes that gripping connectors 54 are attached to target connector 56 at the center portions thereof, and the lateral most ends of the left and right gripping connectors 54 are adjacent one another on the target connector 56. The minimum circumference therefore is the width of the waist portion plus the width of each tab from the lateral exterior of the waist portion to the gripping connectors, plus the width of each gripping connector. The minimum circumference in the stretched condition also does not take into consideration the stretchability of target connector 56, since the waist region 400 where target connector 56 is disposed is not stretched. The minimum circumference can therefore be calculated as follows:

Minimum Circumference: (circumference of the waist of article 20 when both gripping connectors 54 are positioned at the center of target connector 56):

Unstretched Min. Circumference=width L +distance from (Right+Left) edges 320, 340, 350, or 360 to inside edge of hook/tape (unstretched)+2×width of gripping connector 54.

Stretched Min. Circumference=width L′+distance from (Right+Left) edges 320, 340, 350, or 360 to inside edge of hook/tape (stretched)+2×width of gripping connector 54.

From the foregoing calculations, the stretchability of the backsheet of article 20 can easily be obtained. It is simply L′−L. By stretchability of backsheet 22, it is understood to mean the stretchability of the overall article 20, but it typically is the backsheet 22 material that limits the degree to which article 20 can stretch in the lateral direction (98). After calculating the backsheet stretchability, one can readily calculate the stretchability of the connection mechanisms 52 by subtracting the backsheet stretchability from the overall stretching of the portion of article 20 from the edge of each tab connection mechanism 52, as follows:

Tab Stretchability=(Tab−Tab Stretch Width)−(Tab−Tab Unstretched Width)−(L−L′).

Those skilled in the art will appreciate that the tab stretchability is the combined stretchability of both right and left tabs, or of one connection mechanism 52, if a one-piece belt-like mechanism were employed.

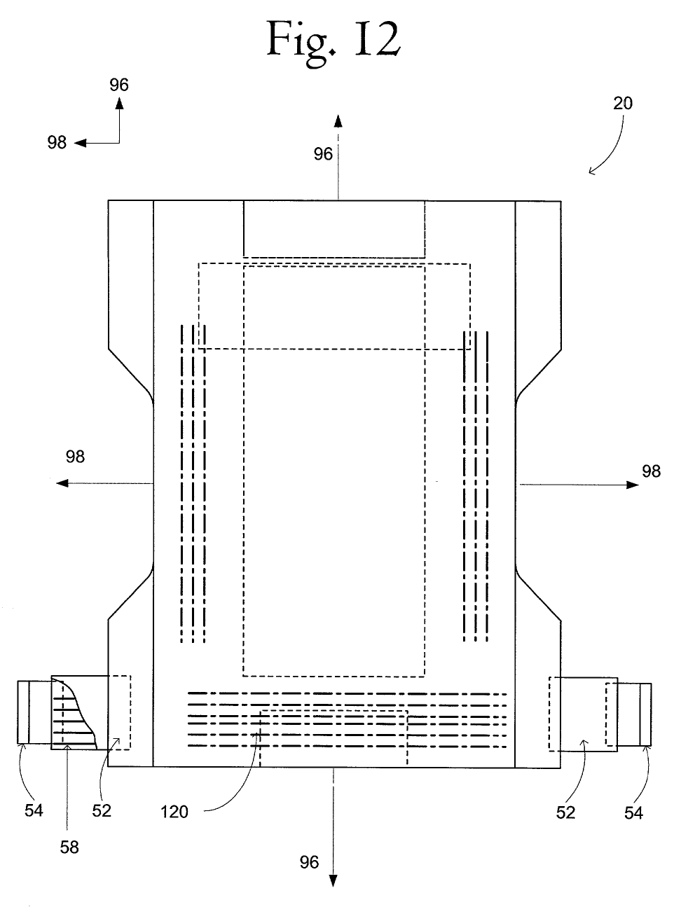

One can impart additional stretchability to backsheet 22 of article 20 in any number of ways, as described above. FIG. 12 illustrates article 20 whereby backsheet 22 has elastic elements 120 either disposed or intertwined therein, or otherwise associated therewith. Any number and size of elastic elements can be employed so long as they impart the desired stretchability. FIG. 12 also illustrates a cut-away portion of connection mechanism 52 showing elastic elements 58 disposed therein (see also FIGS. 7 and 8).

It is preferred in the present invention to render backsheet 22 and connection mechanism 52 stretchable by elastomeric non-woven tabs and foam in the backsheet 22.

The above-mentioned dimensions and stretching tests typically are conducted on a number of articles having various sizes, or stages. Overall stretching characteristics for groups of stages then can be determined based on the average of the stretching characteristics taken over the range of stages tested. For example, for absorbent articles for newborn babies, stages 0, 1, and 2, it is preferred in the present invention that the maximum stretchability of the circumference of the waist region that contains the connection mechanisms is greater than about 185%, whereby the maximum stretchability of the circumference of the waist region is determined by the following equation:

$$Max Stretch = \frac{{\sum }_{n = 1}^{n} ( Max Cirst / MnCirun ) \times 100}{n}$$

wherein:

MxCirstr is the stretched maximum circumference determined in accordance with the equation above;

MnCirun is the unstretched minimum circumference determined in accordance with the equation above; and

n is the number of stages of diapers in which the maximum stretchability is measured.

For absorbent articles for crawling babies, stages 3 and 4, it is preferred in the present invention that the maximum stretchability is greater than about 175%, and for walking babies, stages 5 and 6, it is preferred in the present invention that the maximum stretchability is greater than about 175%. Thus, for the 800 gram force, there will be a MaxStretch value for the newborn babies, crawling babies, and walking babies stages of the garment tested, whereby the MaxStretch is essentially the average of the Maximum Stretchabilities over the range of stages. There will be similar values for the 1,000 gram force and the 1,200 gram force. For all of these tests, it is preferred that MaxStretch be greater than 185%, more preferably greater than 190% and most preferably greater than 195%.

A feature attributable to the full range of garments tested under each force and variety of stages described herein (e.g., the 800 gram force, the 1,000 gram force, and the 1,200 gram force) is the Stretchability Index (or SI). SI is a measure of how well the article will stretch over its full range of sizes, when subjected to the three forces (or loads; 800, 1,000, and 1,200 grams) described herein. SI provides a suitable measure of an absorbent article's stretching ability under loads that typically will be encountered when in use. SI is simply the sum of the MaxStretch values for each force:

SI=MaxStretch₍₈₀₀₎+MaxStretch_{(1,000)}+MaxStretch_{(1,200)}

wherein:

MaxStretch₍₈₀₀₎ is MaxStretch for the 800 gram force;

MaxStretch_{(1,000)} is MaxStretch for the 1,000 gram force; and

MaxStretch_{(1,200)} is MaxStretch for the 1,200 gram force.

Thus, there will be a Stretchability Index for newborns (stages 0, 1, and 2), for crawling babies (stages 3 and 4), and for walking babies (stages 5 and 6). It is preferred in the present invention that the Stretchability Index of newborn baby garments be greater than about 540, more preferably, greater than about 545, and most preferably, greater than about 550. It also is preferred that the Stretchability Index of crawling baby garments be greater than about 515, more preferably, greater than about 520, and most preferably, greater than about 525, and the Stretchability Index of walking baby garments be greater than about 505, more preferably, greater than about 515, and most preferably, greater than about 525.

It also is preferred in the present invention, that for the 800 gram force, the backsheet 22 have a stretchability, or % Bst (percent stretchability of the backsheet), of greater than about 125%, preferably greater than about 130% and most preferably, greater than about 140%. The stretchability of backsheet 22 is simply (L′/L)×100%, which represents the amount the material stretches relative to its initial, unstretched dimension. It is preferred in the present invention, that for the 1,000 gram force, the backsheet 22 have a stretchability of greater than about 130%, preferably greater than about 135% and most preferably, greater than about 140%. In addition, it is preferred in the present invention, that for the 1,200 gram force, the backsheet 22 have a stretchability of greater than about 130%, preferably greater than about 135% and most preferably, greater than about 140%.

The connection mechanisms 52 also preferably has a stretchability, or % Tst (percent stretchability of the tab), for the 800 gram force that is greater than about 200%, more preferably greater than about 210%, and most preferably greater than about 215%. The stretchability of the connection mechanism 52 is simply the (stretched length of the tab/unstretched length of the tab)×100%, or the amount the connection mechanism stretches, based on its initial, unstretched dimension. It is preferred in the present invention, that for the 1,000 gram force, the connection mechanism 52 have a stretchability of greater than about 200%, preferably greater than about 210% and most preferably, greater than about 215%. In addition, it is preferred in the present invention, that for the 1,200 gram force, the connection mechanism 52 have a stretchability of greater than about 210%, preferably greater than about 215% and most preferably, greater than about 218%.

The invention will now be explained with reference to particularly preferred non-limiting examples and comparative examples, as follows.

### EXAMPLES

In the following examples, a number of diapers were purchased from a variety of diaper manufacturers. These diapers then were subjected to the testing protocol outlined in more detail below.

The 800 Gram Force Test

A series of diapers were purchased in each size category, stages 1 through 6. Each letter in the table below designates a particular type of diaper, and that letter is used consistently throughout the examples. For example, the letter A may designate Paragon Trade Brands (PTB) Supremes diapers, the letter B may designate PTB Ultra 3.5 diapers, and the like. The diapers were purchased and tested in accordance with the procedures outlined above. Each of the aforementioned diapers in each size category was unpackaged, laid flat, and the width of the tab connectors and the target connectors were measured and recorded. The diapers then were cut laterally at about 4 inches from the waist edge adjacent the tab connectors as shown in FIG. 2, laid flat by placing weights thereon, and the widths of the portion of the article L, and various dimensions of the tabs were measured as shown in FIGS. 3, 4, 5, and 6.

Each of the aforementioned diaper portions then were clamped to an apparatus 10, such as that shown in FIG. 10, and then attached to an 800 gram weight, as shown in FIG. 11. After gently lowering the cut portion, and waiting about 30 seconds, the dimensions of the stretched portion of the diaper then were measured and recorded. From the recorded dimensions, the backsheet stretchability, the tab stretchability, the maximum stretched circumference, and the minimum unstretched circumference were calculated. The Tab stretchability reported in the tables below is the overall tab stretchability calculated in accordance with the equation above, divided by two (divide by two because there are two tabs). The results for all diapers tested are found in Tables 1-6 below, for the six sizes of diapers (stages 1-6).

In the tables below, the following abbreviations apply:

Bkst=Backsheet stretch in mm;

% Bkst=Percentage of Backsheet stretch calculated by dividing the stretched backsheet width by the unstretched backsheet width and multiplying by 100;

Tst=Tab stretch in mm;

% Tst=Percentage of Tab stretch calculated by dividing Tst by the length of the tab and multiplying by 100

MxCir=Maximum Circumference of stretched article in mm; and

MnCir—Minimum Circumference of unstretched article in mm.

TABLE 1
STAGE 1

| Sample | Bkst | % Bkst | Tst | % Tst | MxCir | MnCir |
|---|---|---|---|---|---|---|
| A | 41 | 119 | 0 | 0 | 458 | 297 |
| B | 29 | 115 | 5 | 119 | 436 | 282 |
| C | 14 | 106 | 14 | 150 | 476 | 325 |
| D | 19 | 108 | 0 | 0 | 458 | 302 |
| E | 52 | 123 | 0 | 0 | 486 | 278 |
| F | 34 | 114 | 0 | 0 | 503 | 302 |
| G | 34 | 117 | 8.5 | 126 | 509 | 292 |
| H | 27 | 111 | 3.5 | 117 | 529 | 362 |
| I | 40 | 121 | 5 | 122 | 433 | 267 |
| J | 27 | 113 | 15 | 154 | 458 | 293 |
| K | 27 | 112 | 0 | 0 | 468 | 306 |
| L | 31 | 113 | 0 | 0 | 474 | 306 |

TABLE 2
STAGE 2

| Sample | Bkst | % Bkst | Tst | % Tst | MxCir | MnCir |
|---|---|---|---|---|---|---|
| A | 51 | 122 | 0 | 0 | 497 | 320 |
| B | 43 | 120 | 7 | 126 | 469 | 298 |
| C | 24 | 110 | 12 | 140 | 574 | 354 |
| D | 27 | 110 | 0 | 0 | 566 | 386 |
| E | 52 | 121 | 0 | 0 | 514 | 312 |
| F | 30 | 112 | 0 | 0 | 521 | 325 |
| G | 43 | 120 | 5 | 119 | 552 | 297 |
| H | 26 | 109 | 0 | 0 | 510 | 328 |
| I | 48 | 123 | 4.5 | 114 | 479 | 305 |
| J | 37 | 118 | 14 | 142 | 496 | 319 |
| K | 31 | 111 | 1 | 105 | 509 | 340 |
| L | 31 | 111 | 0 | 0 | 513 | 346 |

TABLE 3
STAGE 3

| Sample | Bkst | % Bkst | Tst | % Tst | MxCir | MnCir |
|---|---|---|---|---|---|---|
| A | 44 | 117 | 0 | 0 | 534 | 330 |
| B | 44 | 118 | 3.5 | 111 | 544 | 343 |
| C | 18 | 107 | 16 | 146 | 550 | 378 |
| D | 35 | 112 | 0 | 0 | 605 | 406 |
| F | 41 | 114 | 0 | 0 | 562 | 353 |
| G | 56 | 124 | 9 | 130 | 564 | 323 |
| H | 43 | 115 | 0 | 0 | 606 | 405 |
| E | 68 | 125 | 0 | 0 | 549 | 323 |
| I | 49 | 120 | 4 | 113 | 547 | 340 |
| J | 45 | 119 | 9 | 127 | 537 | 343 |
| K | 10 | 105 | 56 | 197 | 571 | 338 |
| M | 35 | 112 | 4 | 118 | 575 | 367 |
| N | 49 | 120 | 8.5 | 129 | 564 | 332 |
| L | 41 | 114 | 0 | 0 | 556 | 361 |

TABLE 4
STAGE 4

| Sample | Bkst | % Bkst | Tst | % Tst | MxCir | MnCir |
|---|---|---|---|---|---|---|
| A | 56 | 120 | 0 | 0 | 559 | 347 |
| B | 46 | 117 | 5 | 118 | 553 | 352 |
| C | 26 | 109 | 9.5 | 128 | 562 | 392 |
| D | 26 | 108 | 0 | 0 | 625 | 403 |
| O | 46 | 116 | 0 | 0 | 575 | 362 |
| P | 50 | 119 | 11 | 134 | 591 | 355 |
| H | 35 | 112 | 0 | 0 | 643 | 432 |
| Q | 60 | 120 | 0 | 0 | 593 | 352 |
| I | 53 | 121 | 4.5 | 114 | 561 | 353 |
| J | 36 | 115 | 11 | 133 | 535 | 349 |
| K | 13 | 107 | 54.5 | 178 | 575 | 343 |
| F | 43 | 114 | 2.5 | 111 | 590 | 375 |
| G | 54 | 121 | 9 | 128 | 584 | 347 |
| L | 40 | 114 | 0 | 0 | 567 | 356 |

TABLE 5
STAGE 5

| Sample | Bkst | % Bkst | Tst | % Tst | MxCir | MnCir |
|---|---|---|---|---|---|---|
| A | 56 | 119 | 0 | 0 | 581 | 366 |
| B | 57 | 121 | 4 | 113 | 572 | 359 |
| C | 22 | 108 | 10 | 130 | 596 | 404 |
| D | 44 | 114 | 0 | 0 | 651 | 414 |
| F | 39 | 112 | 0 | 0 | 589 | 383 |
| G | 55 | 121 | 8.5 | 126 | 603 | 363 |
| H | 28 | 109 | 0 | 0 | 646 | 436 |
| Q | 64 | 121 | 0 | 0 | 589 | 362 |
| I | 51 | 119 | 4 | 114 | 567 | 363 |
| J | 53 | 122 | 8.5 | 124 | 581 | 358 |
| K | 12 | 106 | 52.5 | 173 | 607 | 367 |
| M | 53 | 118 | 3 | 113 | 602 | 377 |
| N | 46 | 117 | 8.5 | 126 | 599 | 370 |

TABLE 6
STAGE 6

| Sample | Bkst | % Bkst | Tst | % Tst | MxCir | MnCir |
|---|---|---|---|---|---|---|
| A | 72 | 124 | 0 | 0 | 594 | 363 |
| B | 51 | 118 | 5 | 118 | 596 | 376 |
| C | 23 | 107 | 9.5 | 133 | 633 | 425 |
| D | 39 | 111 | 0 | 0 | 603 | 449 |
| E | 53 | 116 | 0 | 0 | 606 | 381 |
| F | 54 | 117 | 0 | 0 | 656 | 376 |
| G | 45 | 114 | 0 | 0 | 599 | 449 |
| J | 56 | 121 | 14 | 135 | 633 | 385 |

From the above tables, it can be seen that commercially available diapers do not possess the requisite stretchability using the 800 gram force. That is, all of the diapers presently on the market have a backsheet stretchability less than about 125%, and a tab stretchability of less than about 200%. The elongation of the backsheet for each of the commercially available diapers was less than about 125%, and the Maximum Stretchability for all diapers was less than about 180% (see Table 19 below). The commercially available diapers on the market therefore lack the flexibility to traverse any of the stages to enable their use throughout the size ranges of newborn babies, crawling babies, and walking babies.

The 1,000 Gram Force Test

A series of diapers were purchased in each size category, stages 1 through 6. The diapers were purchased and tested in accordance with the procedures outlined above. Each of the aforementioned diapers in each size category was unpackaged, laid flat, and the width of the tab connectors and the target connectors were measured and recorded. The diapers then were cut laterally at about 4 inches from the waist edge adjacent the tab connectors as shown in FIG. 2, laid flat by placing weights thereon, and the widths of the portion of the article L, and various dimensions of the tabs were measured as shown in FIGS. 3, 4, 5, and 6.

Each of the aforementioned diaper portions then were clamped to an apparatus 10, such as that shown in FIG. 10, and then attached to a 1000 gram weight, as shown in FIG. 11. After gently lowering the cut portion, and waiting about 30 seconds, the dimensions of the stretched portion of the diaper then were measured and recorded. From the recorded dimensions, the backsheet stretchability, the tab stretchability, the maximum stretched circumference, and the minimum unstretched circumference were calculated. The Tab stretchability reported in the tables below is the overall tab stretchability calculated in accordance with the equation above, divided by two (divide by two because there are two tabs). The results for all diapers tested are found in Tables 7-12 below, for the six sizes of diapers (stages 1-6).

In the tables below, the following abbreviations apply:

Bkst=Backsheet stretch in mm;

% Bkst=Percentage of Backsheet stretch calculated by dividing the stretched backsheet width by the unstretched backsheet width and multiplying by 100;

Tst=Tab stretch in mm;

% Tst=Percentage of Tab stretch calculated by dividing Tst by the length of the tab and multiplying by 100

MxCir=Maximum Circumference of stretched article in mm; and

MnCir—Minimum Circumference of unstretched article in mm.

TABLE 7
STAGE 1

| Sample | Bkst | % Bkst | Tst | % Tst | MxCir | MnCir |
|---|---|---|---|---|---|---|
| A | 41 | 119 | 0 | 0 | 460 | 297 |
| B | 28 | 114 | 7.5 | 126 | 441 | 281 |
| C | 27 | 112 | 23 | 185 | 501 | 316 |
| D | 25 | 110 | 0 | 0 | 470 | 307 |
| F | 15 | 106 | 0 | 0 | 509 | 344 |
| G | 30 | 115 | 8 | 124 | 509 | 297 |
| H | 29 | 112 | 0 | 0 | 468 | 305 |
| I | 33 | 117 | 6 | 125 | 436 | 280 |
| J | 46 | 124 | 18.5 | 167 | 476 | 281 |
| K | 38 | 116 | 0 | 0 | 475 | 302 |
| L | 47 | 120 | 0 | 0 | 481 | 297 |

TABLE 8
STAGE 2

| Sample | Bkst | % Bkst | Tst | % Tst | MxCir | MnCir |
|---|---|---|---|---|---|---|
| A | 55 | 124 | 0 | 0 | 481 | 301 |
| B | 43 | 120 | 5 | 119 | 469 | 297 |
| C | 23 | 110 | 11 | 131 | 511 | 360 |
| D | 15 | 105 | 0 | 0 | 520 | 360 |
| F | 38 | 115 | 0 | 0 | 523 | 320 |
| G | 43 | 120 | 5 | 119 | 552 | 297 |
| H | 31 | 110 | 0 | 0 | 520 | 365 |
| I | 42 | 119 | 5 | 116 | 478 | 311 |
| J | 43 | 121 | 15.5 | 144 | 537 | 324 |
| K | 48 | 118 | 0 | 0 | 520 | 336 |
| L | 47 | 117 | 0 | 0 | 517 | 332 |

TABLE 9
STAGE 3

| Sample | Bkst | % Bkst | Tst | % Tst | MxCir | MnCir |
|---|---|---|---|---|---|---|
| A | 45 | 117 | 0 | 0 | 513 | 334 |
| B | 47 | 119 | 4 | 111 | 550 | 347 |
| R | 67 | 125 | 7.5 | 150 | 552 | 335 |
| C | 23 | 109 | 15 | 155 | 549 | 369 |
| D | 30 | 110 | 0 | 0 | 562 | 371 |
| F | 37 | 112 | 0 | 0 | 571 | 367 |
| G | 54 | 123 | 9.5 | 130 | 569 | 329 |
| H | 31 | 110 | 0 | 0 | 546 | 364 |
| E | 68 | 125 | 0 | 0 | 551 | 337 |
| I | 45 | 118 | 4 | 112 | 545 | 344 |
| J | 56 | 125 | 17 | 147 | 559 | 326 |
| S | 56 | 120 | 0 | 0 | 543 | 331 |
| M | 46 | 116 | 0 | 0 | 572 | 361 |
| N | 47 | 119 | 9 | 129 | 570 | 338 |
| K | 37 | 112 | 0 | 0 | 557 | 369 |
| L | 59 | 120 | 0 | 0 | 568 | 357 |

TABLE 10
STAGE 4

| Sample | Bkst | % Bkst | Tst | % Tst | MxCir | MnCir |
|---|---|---|---|---|---|---|
| A | 52 | 118 | 0 | 0 | 565 | 349 |
| B | 48 | 118 | 4 | 116 | 565 | 363 |
| R | 51 | 117 | 6 | 135 | 575 | 378 |
| C | 22 | 108 | 13.5 | 142 | 563 | 391 |
| D | 26 | 108 | 0 | 0 | 581 | 361 |
| T | 32 | 110 | 0 | 0 | 572 | 374 |
| U | 32 | 111 | 0 | 0 | 551 | 391 |
| H | 24 | 107 | 0 | 0 | 593 | 390 |
| Q | 30 | 110 | 0 | 0 | 571 | 363 |
| I | 48 | 118 | 4 | 113 | 569 | 368 |
| J | 48 | 120 | 11.5 | 137 | 542 | 345 |
| K | 13 | 107 | 58.5 | 184 | 602 | 362 |
| F | 51 | 118 | 0 | 0 | 578 | 361 |
| G | 47 | 118 | 8.5 | 128 | 577 | 349 |
| L | 46 | 115 | 0 | 0 | 579 | 409 |

TABLE 11
STAGE 5

| Sample | Bkst | % Bkst | Tst | % Tst | MxCir | MnCir |
|---|---|---|---|---|---|---|
| A | 61 | 121 | 0 | 0 | 581 | 361 |
| B | 46 | 116 | 4 | 114 | 570 | 369 |
| C | 36 | 112 | 17 | 154 | 622 | 406 |
| D | 14 | 104 | 0 | 0 | 608 | 404 |
| F | 21 | 106 | 0 | 0 | 591 | 392 |
| G | 45 | 116 | 8 | 124 | 611 | 383 |
| H | 25 | 107 | 0 | 0 | 600 | 393 |
| Q | 44 | 113 | 0 | 0 | 611 | 386 |
| I | 46 | 116 | 4.5 | 114 | 578 | 379 |
| J | 61 | 125 | 11.5 | 129 | 598 | 361 |
| K | 10 | 105 | 56 | 178 | 624 | 382 |
| S | 60 | 120 | 0 | 0 | 653 | 413 |
| M | 75 | 126 | 0 | 0 | 605 | 365 |
| N | 56 | 121 | 7.5 | 122 | 604 | 369 |

TABLE 12
STAGE 6

| Sample | Bkst | % Bkst | Tst | % Tst | MxCir | MnCir |
|---|---|---|---|---|---|---|
| A | 66 | 122 | 0 | 0 | 598 | 375 |
| B | 56 | 119 | 4.5 | 117 | 608 | 377 |
| C | 28 | 109 | 11.5 | 135 | 631 | 415 |
| D | 37 | 111 | 0 | 0 | 643 | 403 |
| F | 25 | 107 | 0 | 0 | 660 | 410 |
| G | 41 | 113 | 5 | 119 | 632 | 391 |
| H | 27 | 108 | 0 | 0 | 644 | 411 |
| J | 66 | 125 | 15.5 | 140 | 646 | 385 |

From the above tables, it can be seen that commercially available diapers do not possess the requisite stretchability for the 1,000 gram force. That is, all of the diapers presently on the market have a backsheet stretchability less than about 130%, a tab stretchability of less than about 200%. The elongation of the backsheet for each of the commercially available diapers was less than about 130%, and the Maximum Stretchability for all diapers was less than about 180% (see Table 19 below). The commercially available diapers on the market therefore lack the flexibility to traverse any of the stages to enable their use throughout the size ranges of newborn babies, crawling babies, and walking babies.

The 1,200 Gram Force Test

A series of diapers were purchased in each size category, stages 1 through 6. The diapers were purchased and tested in accordance with the procedures outlined above. Each of the aforementioned diapers in each size category was unpackaged, laid flat, and the width of the tab connectors and the target connectors were measured and recorded. The diapers then were cut laterally at about 4 inches from the waist edge adjacent the tab connectors as shown in FIG. 2, laid flat by placing weights thereon, and the widths of the portion of the article L, and various dimensions of the tabs were measured as shown in FIGS. 3, 4, 5, and 6.

Each of the aforementioned diaper portions then were clamped to an apparatus 10, such as that shown in FIG. 10, and then attached to a 1000 gram weight, as shown in FIG. 11. After gently lowering the cut portion, and waiting about 30 seconds, the dimensions of the stretched portion of the diaper then were measured and recorded. From the recorded dimensions, the backsheet stretchability, the tab stretchability, the maximum stretched circumference, and the minimum unstretched circumference were calculated. The Tab stretchability reported in the tables below is the overall tab stretchability calculated in accordance with the equation above, divided by two (divide by two because there are two tabs). The results for all diapers tested are found in Tables 13-18 below, for the six sizes of diapers (stages 1-6).

In the tables below, the following abbreviations apply:

Bkst=Backsheet stretch in mm;

% Bkst=Percentage of Backsheet stretch calculated by dividing the stretched backsheet width by the unstretched backsheet width and multiplying by 100;

Tst=Tab stretch in mm;

% Tst=Percentage of Tab stretch calculated by dividing Tst by the length of the tab and multiplying by 100

MxCir=Maximum Circumference of stretched article in mm; and

MnCir—Minimum Circumference of unstretched article in mm.

TABLE 13
STAGE 1

| Sample | Bkst | % Bkst | Tst | % Tst | MxCir | MnCir |
|---|---|---|---|---|---|---|
| A | 39 | 117 | 0 | 0 | 527 | 369 |
| B | 36 | 118 | 7.5 | 128 | 450 | 281 |
| C | 29 | 113 | 22 | 173 | 501 | 320 |
| D | 28 | 112 | 0 | 0 | 466 | 299 |
| Q | 64 | 129 | 0 | 0 | 547 | 326 |
| F | 50 | 122 | 0 | 0 | 515 | 296 |
| G | 35 | 117 | 10 | 131 | 515 | 293 |
| H | 34 | 114 | 0 | 0 | 477 | 304 |
| I | 72 | 144 | 7 | 129 | 443 | 239 |
| J | 35 | 117 | 19 | 170 | 469 | 288 |
| K | 39 | 116 | 0 | 0 | 481 | 305 |
| L | 40 | 117 | 0 | 0 | 485 | 306 |

TABLE 14
STAGE 2

| Sample | Bkst | % Bkst | Tst | % Tst | MxCir | MnCir |
|---|---|---|---|---|---|---|
| A | 51 | 122 | 0 | 0 | 541 | 362 |
| B | 52 | 124 | 7.5 | 133 | 477 | 293 |
| C | 37 | 115 | 17 | 158 | 536 | 349 |
| D | 33 | 112 | 0 | 0 | 528 | 338 |
| E | 58 | 123 | 0 | 0 | 579 | 370 |
| F | 41 | 116 | 0 | 0 | 607 | 319 |
| H | 24 | 108 | 0 | 0 | 517 | 335 |
| I | 50 | 124 | 7.5 | 123 | 487 | 302 |
| J | 35 | 116 | 19.5 | 158 | 513 | 323 |
| K | 51 | 118 | 0 | 0 | 365 | 312 |
| L | 43 | 115 | 0 | 0 | 526 | 345 |

TABLE 15
STAGE 3

| Sample | Bkst | % Bkst | Tst | % Tst | MxCir | MnCir |
|---|---|---|---|---|---|---|
| A | 48 | 118 | 0 | 0 | 600 | 390 |
| B | 59 | 124 | 5 | 115 | 562 | 342 |
| C | 26 | 110 | 20 | 160 | 566 | 375 |
| D | 40 | 113 | 0 | 0 | 569 | 361 |
| F | 43 | 114 | 0 | 0 | 575 | 363 |
| G | 52 | 122 | 11 | 135 | 566 | 331 |
| H | 46 | 115 | 0 | 0 | 560 | 353 |
| E | 68 | 129 | 0 | 0 | 603 | 376 |
| I | 47 | 118 | 6.5 | 122 | 551 | 341 |
| J | 33 | 113 | 18.5 | 154 | 561 | 359 |
| K | 47 | 116 | 0 | 0 | 565 | 364 |
| M | 45 | 115 | 0 | 0 | 576 | 365 |
| L | 51 | 117 | 0 | 0 | 569 | 363 |

TABLE 16
STAGE 4

| Sample | Bkst | % Bkst | Tst | % Tst | MxCir | MnCir |
|---|---|---|---|---|---|---|
| A | 60 | 121 | 0 | 0 | 633 | 421 |
| B | 53 | 120 | 7.5 | 122 | 580 | 362 |
| R | 61 | 120 | 7 | 145 | 588 | 375 |
| C | 30 | 111 | 17 | 155 | 575 | 383 |
| D | 38 | 113 | 0 | 0 | 626 | 410 |
| T | 52 | 118 | 0 | 0 | 572 | 352 |
| U | 50 | 119 | 21 | 164 | 596 | 360 |
| H | 39 | 112 | 0 | 0 | 590 | 370 |
| Q | 48 | 116 | 0 | 0 | 628 | 376 |
| I | 62 | 124 | 6.5 | 119 | 578 | 355 |
| K | 12 | 106 | 69.5 | 205 | 626 | 364 |
| F | 63 | 122 | 2 | 109 | 593 | 358 |

TABLE 17
STAGE 5

| Sample | Bkst | % Bkst | Tst | % Tst | MxCir | MnCir |
|---|---|---|---|---|---|---|
| A | 56 | 119 | 0 | 0 | 643 | 432 |
| B | 57 | 121 | 5.5 | 118 | 578 | 361 |
| C | 27 | 109 | 18.5 | 159 | 618 | 402 |
| D | 39 | 112 | 0 | 0 | 614 | 381 |
| F | 50 | 116 | 0 | 0 | 595 | 377 |
| G | 61 | 123 | 9.5 | 130 | 608 | 366 |
| H | 37 | 111 | 0 | 0 | 600 | 379 |
| Q | 57 | 119 | 0 | 0 | 630 | 410 |
| I | 54 | 119 | 7 | 123 | 583 | 365 |
| J | 48 | 119 | 17.5 | 147 | 604 | 367 |
| K | 18 | 108 | 69.5 | 189 | 676 | 397 |
| M | 70 | 123 | 5.5 | 126 | 619 | 371 |
| N | 56 | 121 | 7.5 | 122 | 604 | 369 |

TABLE 18
STAGE 6

| Sample | Bkst | % Bkst | Tst | % Tst | MxCir | MnCir |
|---|---|---|---|---|---|---|
| A | 66 | 122 | 0 | 0 | 664 | 444 |
| B | 66 | 123 | 6 | 121 | 613 | 370 |
| C | 35 | 111 | 15 | 143 | 652 | 422 |
| D | 49 | 114 | 0 | 0 | 671 | 405 |
| E | 53 | 116 | 0 | 0 | 670 | 436 |
| F | 61 | 120 | 0 | 0 | 665 | 376 |
| G | 47 | 114 | 0 | 0 | 652 | 400 |
| I | 47 | 117 | 21.5 | 155 | 649 | 398 |

From the above tables, it can be seen that commercially available diapers do not possess the requisite stretchability for the 1,200 gram force. That is, all of the diapers presently on the market have a backsheet stretchability less than about 130%, a tab stretchability of less than about 210%. The elongation of the backsheet for each of the commercially available diapers was less than about 130%, and the Maximum Stretchability for all diapers was less than about 180% (see Table 19 below). The commercially available diapers on the market therefore lack the flexibility to traverse any of the stages to enable their use throughout the size ranges for newborn babies, crawling babies, and walking babies.

Maximum Stretchability and Stretchability Index

The Maximum Stretchability and the Stretchability Index was calculated for each of the diapers using the 800, 1,000, and 1,200 gram force tests above. The results are tabulated in Tables 19, 20, and 21 below. Table 19 is for the newborn babies, Table 20 for crawling babies, and Table 21 for walking babies.

TABLE 19
Newborn Babies

| | Maximum Stretchability | | | |
|---|---|---|---|---|
| Sample | 800 | 1000 | 1200 | SI |
| A | 154.8 | 157.3 | 146.1 | 458.2 |
| B | 156 | 157.4 | 161.5 | 474.9 |
| C | 154.3 | 150.2 | 155.1 | 459.6 |
| D | 149.1 | 148.8 | 156 | 453.9 |
| E | 169.8 | — | 162.1 | — |
| F | 163.4 | 155.7 | 182.1 | 501.2 |
| G | 180.1 | 178.6 | 175.8 | 534.5 |
| H | 150.8 | 152.7 | 155.6 | 459.1 |
| I | 159.6 | 154.7 | 173.3 | 487.6 |
| J | 155.9 | 167.6 | 160.8 | 484.3 |
| K | 151.3 | 156 | 153.4 | 460.7 |
| L | 151.6 | 158.8 | 155.5 | 465.9 |

TABLE 20
Crawling Babies

| | Maximum Stretchability | | | |
|---|---|---|---|---|
| Sample | 800 | 1000 | 1200 | SI |
| A | 161.5 | 157.7 | 152.1 | 471.3 |
| B | 157.9 | 157.1 | 162.3 | 477.3 |
| C | 144.4 | 146.4 | 150.5 | 441.3 |
| D | 152.1 | 156.2 | 155.2 | 463.5 |
| E | 169.2 | 160.4 | 163.7 | 493.3 |
| F | 158.3 | 157.8 | 162 | 478.1 |
| G | 171.5 | 169.1 | 168.3 | 508.9 |
| H | 149.2 | 151 | 159 | 459.2 |
| I | 159.9 | 156.5 | 162.2 | 478.6 |
| J | 154.9 | 164.3 | 156.3 | 475.5 |
| K | 168.3 | 158.6 | 163.6 | 490.5 |
| L | 156.6 | 150.3 | 156.7 | 463.6 |

TABLE 21
Walking Babies

| | Maximum Stretchability | | | |
|---|---|---|---|---|
| Sample | 800 | 1000 | 1200 | SI |
| A | 161.9 | 160.2 | 149.2 | 471.3 |
| B | 158.9 | 157.9 | 162.9 | 479.7 |
| C | 148.2 | 152.6 | 154.1 | 454.9 |
| D | 145.8 | 155 | 163.4 | 464.2 |
| E | 160.9 | 158.3 | 153.7 | 472.9 |
| F | 164.1 | 155.9 | 167.3 | 487.3 |
| G | 166.1 | 160.6 | 166.1 | 492.8 |
| H | 140.8 | 154.7 | 160.7 | 456.2 |
| I | 156.2 | 152.5 | 159.7 | 468.4 |
| J | 163.4 | 166.7 | 163.8 | 493.9 |
| K | 165.4 | 163.4 | 170.3 | 499.1 |

As seen from the above tables, the Maximum Stretchability for conventional diapers is less than about 185% for newborn babies for any of the 800 gram, 1,000 gram, or 1,2000 gram forces, and the Maximum Stretchability for conventional diapers is less than about 175% for crawling babies and for walking babies for any of the 800 gram, 1,000 gram, or 1,2000 gram forces. The Stretchability Index for conventional absorbent diapers is less than about 540 for newborn babies, less than about 515 for crawling babies, and less than about 505 for walking babies.

### EXAMPLES

A prototype diaper was prepared from an Ultra 6, Stage 4 diaper (e.g., “crawling”) available from Paragon Trade Brands, Norcross, Ga. The diaper was modified by removing the fastening elements 52 (tabs) and replacing them with a 65 mm×100 mm long tab that was adhered to the backsheet. The tab was comprised of scrim elastic between outer non-woven sheets, a Conwed Plastics Netting material available as product number 750020-008, from Conwed Plastics, Minneapolis, Minn. This scrim material also was disposed between the topsheet and an additional non-woven material in the ear portions 42 of the diaper (see, FIG. 1). The backsheet also was modified by placing an elastomeric foam material between a non-woven material and the topsheet. The prototype diaper are subjected to the same 800, 1,000 and 1,200 gram force testing and measurements as set out in the comparative examples above. The following table 22 summarize the results.

TABLE 22

| Feature | Force | | |
|---|---|---|---|
| | 800 | 1000 | 1200 |
| Bkst | 107 | 125 | 136 |
| % Bkst | 153.5 | 162.5 | 168 |
| Tst | 72.5 | 74 | 76 |
| % Tst | 268.5 | 274.1 | 281.4 |
| MxCir | 622 | 651 | 666 |
| MnCir | 280 | 280 | 280 |
| MaxStr* | 222 | 232.5 | 237.9 |
| SI | | 692.4 | |

*MaxStr determined where n = 1 since only one stage diaper was tested.

Absorbent articles that have a backsheet elongation greater than about 125%, a tab stretchability of greater than 200%, a maximum stretchability well above than about 185%, and a Stretchability Index greater than 540. Indeed, the maximum circumference of the Stage 4 diaper of the present examples is on par with the Stage 6 diapers tested in the comparative examples. Diapers made in accordance with the present invention therefore have improved flexibility and are capable of fitting a wider range of wearers.

While the invention has been described with reference to particularly preferred embodiments and examples, those skilled in the art will appreciate that various modifications may be made thereto without significantly departing from the spirit and scope thereof.

What is claimed is:

1) An absorbent article having a lateral dimension and a longitudinal dimension comprising
a topsheet having a longitudinal front and back end;
a backsheet having a longitudinal front and back end;
an absorbent material disposed between the topsheet and the backsheet;
a back waist edge adjacent the longitudinal back end of the topsheet and backsheet;
a front waist edge adjacent the longitudinal front end of the topsheet and backsheet;
side edges disposed between the back waist edge and the front waist edge;
a back waist region defined by the back waist edge and the side edges;
a front waist region defined by the front waist edge and the side edges;
a crotch region disposed between the back waist region and the front waist region; and
a connection mechanism disposed adjacent at least the front waist region or the back waist region,
whereby the waist region that includes the connection mechanism stretches by more than 125% of its original width, when an 800 gram force is applied to an about 4-inch wide laterally cut portion of the waist region.

2) The absorbent article as claimed in claim 1, whereby the absorbent article comprises two connection mechanisms.

3) The absorbent article as claimed in claim 2, wherein the connection mechanisms further comprise gripping connectors.

4) The absorbent article as claimed in claim 3, wherein the gripping connectors are comprised of a plurality of hooks or loops.

5) The absorbent article as claimed in claim 3, wherein the gripping connectors are comprised of a tape.

6) The absorbent article as claimed in claim 2, wherein at least one of the connection mechanisms stretches by more than about 200% of its original length, when an 800 gram force is applied to an about 4-inch wide laterally cut portion of the waist region.

7) The absorbent article as claimed in claim 1, wherein the waist region stretches by more than about 130% of its original width.

8) The absorbent article as claimed in claim 1, wherein the waist region stretches by more than about 140% of its original width.

9) The absorbent article as claimed in claim 6, wherein the at least one connection mechanism stretches by more than about 210% of its original length.

10) The absorbent article as claimed in claim 6, wherein the at least one connection mechanism stretches by more than about 215% of its original length.

11) The absorbent article as claimed in claim 1, further comprising a target connector having a lateral width disposed in the front waist region, wherein the article comprises at least two connection mechanisms extending outwardly in the lateral dimension from the rear waist region.

12) The absorbent article as claimed in claim 11, wherein the connection mechanisms are tabs, each tab comprising a gripping connector having a width.

13) The absorbent article as claimed in claim 1, wherein the maximum stretchability of the circumference of the waist region for newborn babies is greater than about 185%, whereby the maximum stretchability of the circumference of the waist region is determined by the following equation:
$$Max Stretch = \frac{{\sum }_{n = 1}^{n} ( Max Cirst / MnCirun ) \times 100}{n}$$
wherein:
MxCirstr is the stretched maximum circumference;
MnCirun is the unstretched minimum circumference; and
n is the number of stages of diapers in which the maximum stretchability is measured.

14) The absorbent article as claimed in claim 13, wherein the maximum stretchability of the circumference of the waist region for crawling babies is greater than about 175%.

15) The absorbent article as claimed in claim 13, wherein the maximum stretchability of the circumference of the waist region for walking babies is greater than about 175%.

16) The absorbent article as claimed in claim 1, wherein the waist region that includes the connection mechanism stretches by more than 130% of its original width, when a 1,000 gram force is applied to an about 4-inch wide laterally cut portion of the waist region.

17) The absorbent article as claimed in claim 16, wherein the waist region stretches by more than 135%.

18) The absorbent article as claimed in claim 16, wherein the waist region stretches by more than 140%.

19) The absorbent article as claimed in claim 1, wherein at least one of the connection mechanisms stretches by more than about 200% of its original length, when a 1,000 gram force is applied to an about 4-inch wide laterally cut portion of the waist region.

20) The absorbent article as claimed in claim 19, wherein the at least one connection mechanism stretches by more than about 210% of its original length.

21) The absorbent article as claimed in claim 19, wherein the at least one connection mechanism stretches by more than about 215% of its original length.

22) The absorbent article as claimed in claim 1, wherein the waist region that includes the connection mechanism stretches by more than 130% of its original width, when a 1,200 gram force is applied to an about 4-inch wide laterally cut portion of the waist region.

23) The absorbent article as claimed in claim 22, wherein the waist region stretches by more than 135%.

24) The absorbent article as claimed in claim 22, wherein the waist region stretches by more than 140%.

25) The absorbent article as claimed in claim 1, wherein at least one of the connection mechanisms stretches by more than about 210% of its original length, when a 1,200 gram force is applied to an about 4-inch wide laterally cut portion of the waist region.

26) The absorbent article as claimed in claim 25, wherein the at least one connection mechanism stretches by more than about 215% of its original length.

27) The absorbent article as claimed in claim 25, wherein the at least one connection mechanism stretches by more than about 218% of its original length.

28) The absorbent article as claimed in claim 16, wherein the maximum stretchability of the circumference of the waist region for newborn babies is greater than about 185%, whereby the maximum stretchability of the circumference of the waist region is determined by the following equation:
$$Max Stretch = \frac{{\sum }_{n = 1}^{n} ( Max Cirst / MnCirun ) \times 100}{n}$$
wherein:
MxCirstr is the stretched maximum circumference;
MnCirun is the unstretched minimum circumference; and
n is the number of stages of diapers in which the maximum stretchability is measured.

29) The absorbent article as claimed in claim 28, wherein the maximum stretchability of the circumference of the waist region for crawling babies is greater than about 175%.

30) The absorbent article as claimed in claim 28, wherein the maximum stretchability of the circumference of the waist region for walking babies is greater than about 175%.

31) The absorbent article as claimed in claim 22, wherein the maximum stretchability of the circumference of the waist region for newborn babies is greater than about 185%, whereby the maximum stretchability of the circumference of the waist region is determined by the following equation:
$$Max Stretch = \frac{{\sum }_{n = 1}^{n} ( Max Cirst / MnCirun ) \times 100}{n}$$
wherein:
MxCirstr is the stretched maximum circumference;
MnCirun is the unstretched minimum circumference; and
n is the number of stages of diapers in which the maximum stretchability is measured.

32) The absorbent article as claimed in claim 31, wherein the maximum stretchability of the circumference of the waist region for crawling babies is greater than about 175%.

33) The absorbent article as claimed in claim 31, wherein the maximum stretchability of the circumference of the waist region for walking babies is greater than about 175%.

34) The absorbent article as claimed in claim 1, wherein the article is for newborn babies and has a Stretchability Index of greater than about 540, wherein the Stretchability Index is measured in accordance with the following equation:
SI=MaxStretch₍₈₀₀₎+MaxStretch_{(1,000)}+MaxStretch_{(1,200)}
wherein:
MaxStretch₍₈₀₀₎ is MaxStretch for the 800 gram force;
MaxStretch_{(1,000)} is MaxStretch for the 1,000 gram force; and
MaxStretch_{(1,200)} is MaxStretch for the 1,200 gram force,
whereby each of the MaxStretch values are those for newborn babies.

35) The absorbent article as claimed in claim 34, wherein the SI for crawling babies is greater than about 515, whereby each of the MaxStretch values are those for crawling babies.

36) The absorbent article as claimed in claim 34, wherein the SI for walking babies is greater than about 505, whereby each of the MaxStretch values are those for walking babies.

37) An absorbent article having a lateral dimension and a longitudinal dimension comprising
a topsheet having a longitudinal front and back end;
a backsheet having a longitudinal front and back end;
an absorbent material disposed between the topsheet and the backsheet;
a back waist edge adjacent the longitudinal back end of the topsheet and backsheet;
a front waist edge adjacent the longitudinal front end of the topsheet and backsheet;
side edges disposed between the back waist edge and the front waist edge;
a back waist region defined by the back waist edge and the side edges;
a front waist region defined by the front waist edge and the side edges;
a crotch region disposed between the back waist region and the front waist region; and
at least two connection mechanisms disposed adjacent at least the front waist region or the back waist region,
whereby at least one of the connection mechanisms stretches by more than about 200% of its original length, when an 800 gram force is applied to an about 4-inch wide laterally cut portion of the waist region that contains the connection mechanisms.

38) The absorbent article as claimed in claim 37, wherein the connection mechanisms further comprise gripping connectors.

39) The absorbent article as claimed in claim 38, wherein the gripping connectors are comprised of a plurality of hooks or loops.

40) The absorbent article as claimed in claim 39, wherein the gripping connectors are comprised of a tape.

41) The absorbent article as claimed in claim 37, wherein the at least one connection mechanism stretches by more than about 210% of it original length.

42) The absorbent article as claimed in claim 37, wherein the at least one connection mechanism stretches by more than about 215% of it original length.

43) The absorbent article as claimed in claim 37, wherein the waist region that includes the connection mechanism stretches by more than about 125% of its original width, when an 800 gram force is applied to an about 4-inch wide laterally cut portion of the waist region.

44) The absorbent article as claimed in claim 43, wherein the waist region stretches by more than about 130%.

45) The absorbent article as claimed in claim 43, wherein the waist region stretches by more than about 140%.

46) The absorbent article as claimed in claim 37, further comprising a target connector having a lateral width disposed in the front waist region, wherein the article comprises at least two connection mechanisms extending outwardly in the lateral dimension from the rear waist region.

47) The absorbent article as claimed in claim 46, wherein the connection mechanisms are tabs, each tab comprising a gripping connector having a width.

48) The absorbent article as claimed in claim 37, wherein the maximum stretchability of the circumference of the waist region for newborn babies is greater than about 185%, whereby the maximum stretchability of the circumference of the waist region is determined by the following equation:
$$Max Stretch = \frac{{\sum }_{n = 1}^{n} ( Max Cirst / MnCirun ) \times 100}{n}$$
wherein:
MxCirstr is the stretched maximum circumference;
MnCirun is the unstretched minimum circumference; and
n is the number of stages of diapers in which the maximum stretchability is measured.

49) The absorbent article as claimed in claim 48, wherein the maximum stretchability of the circumference of the waist region for crawling babies is greater than about 175%.

50) The absorbent article as claimed in claim 48, wherein the maximum stretchability of the circumference of the waist region for walking babies is greater than about 175%.

51) A method of determining the stretchability of the waist region of an absorbent article having a lateral dimension and a longitudinal dimension, the absorbent article comprising
a topsheet having a longitudinal front and back end;
a backsheet having a longitudinal front and back end;
an absorbent material disposed between the topsheet and the backsheet;
a back waist edge adjacent the longitudinal back end of the topsheet and backsheet;
a front waist edge adjacent the longitudinal front end of the topsheet and backsheet;
side edges disposed between the back waist edge and the front waist edge;
a back waist region defined by the back waist edge and the side edges;
a front waist region defined by the front waist edge and the side edges;
a crotch region disposed between the back waist region and the front waist region; and
a connection mechanism disposed adjacent at least the front waist region or the back waist region
whereby the method comprises:
laterally cutting about a 4-inch longitudinal section of the waist region that includes the connection mechanism;
flattening the longitudinal section of the waist region on a surface without substantially stretching the longitudinal section;
measuring the dimensions of the longitudinal section of the waist region including the dimensions of the connection mechanism;
attaching a first end of the connection mechanism or longitudinal section to a first surface; and
attaching the opposite end of the longitudinal section or a second end of the connection mechanism disposed on the opposite side of the longitudinal section from the first end to a second surface;
applying a predetermined force to the longitudinal section of the waist region including the connection mechanism to thereby stretch the longitudinal section and connection mechanism;
measuring the dimensions of the stretched longitudinal section and connection mechanism;
and calculating the stretchability of the longitudinal section, and the stretchability of the connection mechanism.

52) The method as claimed in claim 51, wherein applying the predetermined force comprises using an Instron device to apply the force.

53) The method as claimed in claim 51, wherein applying the predetermined force comprises suspending a weight from the second surface.

54) The method as claimed in claim 51, wherein the predetermined force is 800 grams.

55) The method as claimed in claim 51, wherein the predetermined force is 1,000 grams.

56) The method as claimed in claim 51, wherein the predetermined force is 1,200 grams.

57) An absorbent article having a lateral dimension and a longitudinal dimension comprising
a topsheet having a longitudinal front and back end;
a backsheet having a longitudinal front and back end;
an absorbent material disposed between the topsheet and the backsheet;
a back waist edge adjacent the longitudinal back end of the topsheet and backsheet;
a front waist edge adjacent the longitudinal front end of the topsheet and backsheet;
side edges disposed between the back waist edge and the front waist edge;
a back waist region defined by the back waist edge and the side edges;
a front waist region defined by the front waist edge and the side edges;
a crotch region disposed between the back waist region and the front waist region; and
a connection mechanism disposed adjacent at least the front waist region or the back waist region,
whereby the waist region that includes the connection mechanism has a maximum stretchability of the circumference thereof for newborn babies of more than 185%, when a force selected from the group consisting of an 800 gram, 1,000 gram, and 1,200 gram is applied to an about 4-inch wide laterally cut portion of the waist region, the maximum stretchability being determined by the following equation:
$$Max Stretch = \frac{{\sum }_{n = 1}^{n} ( Max Cirst / MnCirun ) \times 100}{n}$$
wherein:
MxCirstr is the stretched maximum circumference;
MnCirun is the unstretched minimum circumference; and
n is the number of stages of diapers in which the maximum stretchability is measured.

58) The absorbent article as claimed in claim 57, wherein the maximum stretchability of the circumference of the waist region for crawling babies is greater than about 175%.

59) The absorbent article as claimed in claim 57, wherein the maximum stretchability of the circumference of the waist region for walking babies is greater than about 175%.

60) An absorbent article having a lateral dimension and a longitudinal dimension comprising
a topsheet having a longitudinal front and back end;
a backsheet having a longitudinal front and back end;
an absorbent material disposed between the topsheet and the backsheet;
a back waist edge adjacent the longitudinal back end of the topsheet and backsheet;
a front waist edge adjacent the longitudinal front end of the topsheet and backsheet;
side edges disposed between the back waist edge and the front waist edge;
a back waist region defined by the back waist edge and the side edges;
a front waist region defined by the front waist edge and the side edges;
a crotch region disposed between the back waist region and the front waist region; and
a connection mechanism disposed adjacent at least the front waist region or the back waist region,
whereby the waist region that includes the connection mechanism has a Stretchability Index of greater than about 540, wherein the Stretchability Index is measured in accordance with the following equation:
SI=MaxStretch₍₈₀₀₎+MaxStretch_{(1,000)}+MaxStretch_{(1,200)}
wherein:
MaxStretch₍₈₀₀₎ is MaxStretch for an 800 gram force;
MaxStretch_{(1,000)} is MaxStretch for a 1,000 gram force; and
MaxStretch_{(1,200)} is MaxStretch for a 1,200 gram force,
whereby each of the MaxStretch values are those for newborn babies, and whereby MaxStretch is determined in accordance with the following equation:
$$Max Stretch = \frac{{\sum }_{n = 1}^{n} ( Max Cirst / MnCirun ) \times 100}{n}$$
wherein:
MxCirstr is the stretched maximum circumference;
MnCirun is the unstretched minimum circumference; and
n is the number of stages of diapers in which the maximum stretchability is measured.

61) The absorbent article as claimed in claim 60, wherein the SI for crawling babies is greater than about 515, whereby each of the MaxStretch values are those for crawling babies.

62) The absorbent article as claimed in claim 60, wherein the SI for walking babies is greater than about 505, whereby each of the MaxStretch values are those for walking babies.
